(19) United States
(12) Patent Application Publication
(10) Pub. No.: US 2024/0304313 A1
(43) Pub. Date: Sep. 12, 2024

(54) SYSTEMS AND METHODS FOR REMOTE PATIENT MONITORING

(71) Applicant: Optum Services (Ireland) Limited, Dublin (IE)

(72) Inventors: Damian KELLY, Kilcock (IE); Gregory BUCKLEY, Dublin (IE); Öznur ALKAN, Dublin (IE); Megan O'BRIEN, Naas (IE); Fantine Sylvie MORDELET, Dublin (IE)

(73) Assignee: Optum Services (Ireland) Limited, Dublin (IE)

(21) Appl. No.: 18/182,155

(22) Filed: Mar. 10, 2023

(51) Int. Cl. G16H 40/20; A61B 5/00; G16H 50/30

(52) U.S. Cl. CPC G16H 40/20; A61B 5/0002; A61B 5/74; G16H 50/30

(57) ABSTRACT

Systems and computer-implemented methods for improved provision of health alerts associated with patients are disclosed. A computer-implemented method includes receiving a first reading for a first biometric parameter for a first patient. The method includes applying a plurality of algorithms that determine a plurality of scores, respectively, for the first reading. Each of the plurality of algorithms uses different logic. The method includes determining, using a machine learning model, an aggregate score based on the determined plurality of first scores and on a learned weighting of the plurality of algorithms. The method includes comparing the aggregate score to a threshold. The method includes providing an alert to a user based on the comparing.

### TECHNICAL FIELD

Various embodiments of the present disclosure relate generally to systems and methods for remote patient monitoring, and more particularly to, systems, computer-implemented methods, and non-transitory computer readable mediums for balancing alerting algorithms.

### BACKGROUND

Remote Patient Monitoring (RPM) may provide vital life support and monitoring. Existing RPM alerting technologies use fixed thresholds against which biometric measurements are compared to, ideally, raise alerts early enough to address detected problems. These fixed thresholds may be tied to a unit of the biometric measurement. For example, when a patient's blood sugar recording (e.g., in mg/dL) is measured to be below or above a predefined fixed blood sugar threshold (e.g., in mg/dL), then the system may raise an alert.

However, these fixed thresholds cannot fully capture complex scenarios that happen in real life. For example, a fixed pulse limit of 120 may produce many unnecessary alerts for patients who have a higher heart rate baseline than what is commonly deemed normal. These fixed threshold-based RPM alerting systems may result in a situation called “alert fatigue,” where a health provider's response rate and/or care efficiency may suffer due to receiving excessive alerts. In addition, these systems do not provide comprehensive insights around why an alert is raised, making it hard for a health care provider to decide whether an alert is clinically relevant or not. In addition, existing solutions may process readings from only one biometric type at a time, even when readings from multiple biometric types are available.

Some systems may use default thresholds for a specific biometric (e.g., upper and lower bound for weight for general population) which may be manually raised by a clinician for an individual if required (e.g., if a patient has an abnormally high weight consistently above the default upper bound). However, this manual solution depends on a clinician's ability to spot these abnormal cases, and boundaries may have to be repetitively manually reset as patient conditions change. Furthermore, this may “under-alert” for some patients; for example, if a patient has a very consistent weight record and then has a reading that deviates greatly but is still between thresholds. In this example, such behavior would be concerning, but would not raise an alert by these systems.

Some systems may measure a deviation of a patient's biometric data from their baseline, significant prolonged changes in trend data, or a standard deviation based on measurements over a previous number of days, and raise an alert if the variability is unusually high as compared to prior readings. However, these systems still cannot account for a variety of possible scenarios or unusual behaviors, and have generally been shown to require a significant trade-off between accuracy and alert fatigue.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, computer-implemented methods, systems, and non-transitory computer readable mediums are disclosed for balancing alerting algorithms.

In one aspect, a computer-implemented method for improved provision of health alerts associated with patients is disclosed. The method may include receiving, by one or more processors, a first reading for a first biometric parameter for a first patient. The method may further include, applying, by the one or more processors, a plurality of algorithms that determine a plurality of first scores, respectively, for the first reading. Each of the plurality of algorithms may use different logic. The method may further include, determining, by the one or more processors and using a machine learning model, an aggregate score based on the determined plurality of first scores and a learned weighting of the plurality of algorithms. The method may further include comparing, by the one or more processors, the aggregate score to a threshold. The method may further include providing, by the one or more processors, an alert to a user based on the comparing.

The machine learning model may be trained based at least in part on hospitalization events. Each first score may indicate a probability of hospitalization based on the first reading.

The machine learning model may be trained based at least in part on medical events. The machine learning model may be trained using a plurality of training readings. Each training reading may be assigned a ground truth label based on whether the training reading occurred during a predetermined period of time before a medical event. The predetermined period of time may be a calculated admission window and the medical event may be an admission date to a hospital.

The user may be the first patient or a health care provider. The method may further include providing, by the one or more processors, an explanation for the alert based on the learned weighting of the plurality of algorithms and the aggregate score.

The method may further include ranking, by the one or more processors, the plurality of algorithms based on a contribution of each algorithm to the aggregate score. The method may further include providing a list of algorithms based on the ranking.

The method may further include receiving, by the one or more processors, a second reading for a second biometric parameter for the first patient. The method may further include applying, by the one or more processors, the plurality of algorithms to determine a plurality of second scores, respectively, for the second reading. The determined aggregate score may be further based on the plurality of second scores.

The method may further include receiving, by the one or more processors, additional information for the patient. The aggregate score may be based on the received additional information.

The method may further include receiving, by the one or more processors, a second reading for a second patient. The method may further include applying, by the one or more processors, the plurality of algorithms that determine a plurality of second scores, respectively, to the second reading. The method may further include determining, by the one or more processors and using the machine learning model, a secondary aggregate score for the second patient based on the determined plurality of second scores. The method may further include ranking, by the one or more processors, the aggregate score and the secondary aggregate score. The method may further include providing, by the one or more processors, the aggregate score and the secondary aggregate score based on the ranking.

The threshold may be based on a user input and/or a predetermined alert frequency.

In another aspect, a system for improved provision of health alerts associated with patients is disclosed. The system may include a memory having processor-readable instructions stored therein and a processor configured to access the memory and execute the processor-readable instructions to perform operations. The operations may include receiving a first reading for a first biometric parameter for a first patient. The operations may further include applying a plurality of algorithms that determine a plurality of scores, respectively, for the first reading. Each of the plurality of algorithms may use different logic. The operations may further include determining, using a machine learning model, an aggregate score based on the determined plurality of first scores and on a learned weighting of the plurality of algorithms. The operations may further include comparing the aggregate score to a threshold. The operations may further include providing an alert to a user based on the comparing.

The machine learning model may be trained based at least in part on medical events. Each first score may indicate a probability of hospitalization based on the first reading.

In yet another aspect, a non-transitory computer-readable medium storing a set of instructions that, when executed by a processor, perform operations for improved provision of health alerts associated with patients is disclosed. The operations may include receiving a first reading for a first biometric parameter for a first patient. The operations may further include applying a plurality of algorithms that determine a plurality of scores, respectively, for the first reading. Each of the plurality of algorithms may use different logic. The operations may further include determining, using a machine learning model, an aggregate score based on the determined plurality of first scores and on a learned weighting of the plurality of algorithms. The operations may further include comparing the aggregate score to a threshold. The operations may further include providing an alert to a user based on the comparing.

The machine learning model may be trained based at least in part on medical events. Each first score may indicate a probability of hospitalization based on the first reading. The machine learning model may be trained using a plurality of training readings. Each training reading may be assigned a ground truth label based on whether the training reading occurred during a predetermined period of time before a medical event.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 depicts a block diagram of an exemplary system for balancing alerting algorithms, according to one or more embodiments.

FIG. 2 depicts a flow chart illustrating exemplary processing steps in an exemplary remote patient monitoring (RMS) system, according to an exemplary embodiment.

FIG. 3 depicts an exemplary remote patient monitoring (RMS) system, according to an exemplary embodiment.

FIG. 4 depicts a flow chart illustrating method of training and using an RMS system, according to an exemplary embodiment.

FIG. 5 depicts exemplary reading data for a patient, according to an exemplary embodiment.

FIGS. 6A through 6D depict exemplary displays that analyze biometric reading data using alerting algorithms.

FIG. 7 depicts an exemplary calculated admission window, according to an exemplary embodiment.

FIG. 8 depicts an exemplary model inputs matrix and an exemplary model target matrix to train a machine learning model, according to an exemplary embodiment.

FIG. 9A depicts an exemplary model inputs matrix using additional patient information, according to an exemplary embodiment, and FIG. 9B depicts an exemplary model inputs matrix that uses binary indications for the additional patient information, according to an exemplary embodiment.

FIG. 10 depicts an example of validation results, according to an exemplary embodiment.

FIG. 11 depicts an example of validation results and factors at various thresholds, according to an exemplary embodiment.

FIG. 12A depicts an exemplary user interface in determining a threshold, according to an exemplary embodiment.

FIG. 12B is a flow chart illustrating an exemplary training method, according to an exemplary embodiment.

FIG. 12C is a flow chart illustrating an exemplary training method, according to an exemplary embodiment.

FIG. 13 depicts a method for using a machine learning system to determine whether to raise an alert, according to an exemplary embodiment.

FIG. 14 depicts a method for using the machine learning system of FIG. 13 across multiple patients, according to an exemplary embodiment.

FIG. 15 depicts an exemplary output or display of an analysis of the multiple patients of FIG. 14, according to an exemplary embodiment.

FIGS. 16A and 16B depict exemplary graphical user interfaces of using the machine learning system of FIG. 13, according to an exemplary embodiment.

FIG. 17 is a flow chart illustrating a method to determine an explanation to display on a graphical interface (e.g., FIGS. 16A and 16B), according to an exemplary embodiment.

FIG. 18 is a flow chart illustrating a method of using a machine learning system to predict whether to raise an alert, according to an exemplary embodiment.

FIG. 19 depicts an implementation of a computer system that may execute techniques presented herein.

### DETAILED DESCRIPTION

Various embodiments of the present disclosure relate generally to remote patient monitoring. More particularly, various embodiments of the present disclosure relate to systems, computer-implemented methods, and non-transitory computer readable mediums for balancing or weighting alerting algorithms.

As discussed above, existing RPM alerting systems may over-alert, may not provide much insight into why an alert was raised, and may require a significant tradeoff between over-alerting and accuracy in providing clinically relevant alerts or notifications. There is no one-size-fits-all algorithm.

Motivated from the limitations of the existing RPM alerting systems, techniques disclosed herein may provide an enhanced approach and/or maximize a hospitalization event recall while minimizing alerting frequency. Aspects disclosed herein may provide a personalized, automated, and explainable alerting system that reduces health care provider's alert fatigue through combining different algorithms, each of which is designed to extract different types of risky patterns from data. For example, aspects of the present disclosure may provide for automatic detection of acute patterns in a patient's biometric data and highlight the key patterns that are causing a patient's risk level to be so high that an alert is raised. Aspects of the present disclosure may also provide for detection of a greater amount of complex patterns in a patient's biometric data than existing RPM alerting systems, which may provide comprehensive insight related to predicting a patient's negative health outcome. Such aspects of the present disclosure may improve the prediction accuracy of patient health outcomes.

Aspects disclosed herein may improve clinical relevance of alerts. In addition, aspects disclosed herein may provide a transparent and interactive framework that enables health care providers or institutions to select a desired alert frequency and/or response threshold for identifying risky patterns in an informed manner, which is not offered by existing solutions.

Aspects disclosed herein may use a machine learning model to combine powers of individual alerting algorithms through learning an optimal weighting between them to estimate a need of raising an alert ahead of a hospitalization event. Aspects disclosed herein may provide a machine learning model configured to process readings from multiple biometric types together to reach conclusions accordingly.

The terminology used herein may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In the detailed description herein, references to “embodiment,” “an embodiment,” “one non-limiting embodiment,” “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

In general, terminology can be understood at least in part from usage in context. For example, terms, such as “and”, “or”, or “and/or,” as used herein can include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, can be used to describe any feature, structure, or characteristic in a singular sense or can be used to describe combinations of features, structures or characteristics in a plural sense. Similarly, terms, such as “a,” “an,” or “the,” again, can be understood to convey a singular usage or to convey a plural usage, depending at least in part upon context. In addition, the term “based on” can be understood as not necessarily intended to convey an exclusive set of factors and can, instead, allow for existence of additional factors not necessarily expressly described, again, depending at least in part on context.

As used herein, the terms “comprises,” “comprising,” “includes,” “including,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

The term “clinician” may include, for example, without limitation, any person, organization, and/or collection of persons that provides medical care (i.e., health care provider). For example, a clinician may include a physician, a nurse, a psychologist, an optometrist, a veterinarian, a physiotherapist, a dentist, and a physician assistant.

As used herein, a “machine-learning model” generally encompasses instructions, data, and/or a model configured to receive input, and apply one or more of a weight, bias, classification, or analysis on the input to generate an output. The output may include, for example, an analysis based on the input, a prediction, suggestion, or recommendation associated with the input, a dynamic action performed by a system, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine-learning techniques, such as k-nearest neighbors, linear regression, logistical regression, random forest, gradient boosted machine (GBM), support-vector machine, deep learning, text classifiers, image recognition classifiers, You Only Look Once (YOLO), a deep neural network, greedy matching, propensity score matching, and/or any other suitable machine-learning technique that solves problems specifically addressed in the current disclosure. Supervised, semi-supervised, and/or unsupervised training may be employed. For example, supervised learning may include providing training data and labels corresponding to the training data, e.g., as ground truth. Unsupervised approaches may include clustering, classification, principal component analysis (PCA) or the like. K-means clustering or K-Nearest Neighbors may also be used, which may be supervised or unsupervised. Combinations of K-Nearest Neighbors and an unsupervised cluster technique may also be used. Other models for detecting objects in contents/files, such as documents, images, pictures, drawings, and media files may be used as well. Any suitable type of training may be used, e.g., stochastic, gradient boosted, random seeded, recursive, epoch or batch-based, etc.

Certain non-limiting embodiments are described below with reference to block diagrams and operational illustrations of methods, processes, devices, and apparatus. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

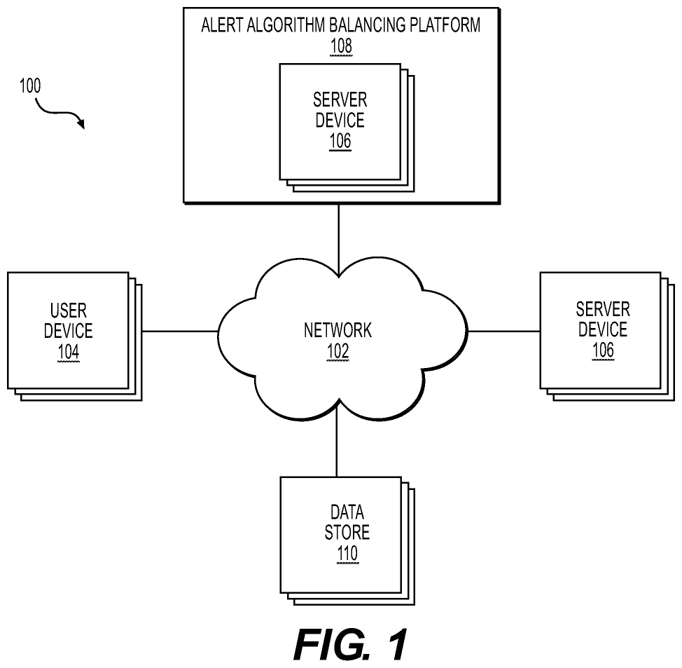

Referring now to the appended drawings, FIG. 1 depicts a block diagram of an exemplary system 100 for balancing alerting algorithms and/or their results, according to one or more embodiments. As illustrated in FIG. 1, the system 100 may include a network 102, one or more user devices 104, one or more server devices 106, an alerting algorithm balancing platform 108, which may include one or more of the server devices 106, and one or more data stores 110.

The network 102 may include a wired and/or wireless network that may couple devices so that communications can be exchanged, such as between a server and a user device or other types of devices, including between wireless devices coupled via a wireless network, for example. A network can also include mass storage, such as network attached storage (NAS), a storage area network (SAN), or other forms of computer or machine-readable media, for example. A network can include the Internet, one or more local area networks (LANs), one or more wide area networks (WANs), wire-line type connections, wireless type connections, cellular or any combination thereof. Likewise, sub-networks, which can employ differing architectures or can be compliant or compatible with differing protocols, can interoperate within a larger network. Various types of devices can, for example, be made available to provide an interoperable capability for differing architectures or protocols. As one illustrative example, a router can provide a link between otherwise separate and independent LANs.

Furthermore, devices or user devices, such as computing devices or other related electronic devices can be remotely coupled to a network, such as via a wired or wireless line or link, for example.

In certain non-limiting embodiments, a “wireless network” should be understood to couple user devices with a network. A wireless network can include virtually any type of wireless communication mechanism by which signals can be communicated between devices, between or within a network, or the like. A wireless network can employ standalone ad-hoc networks, mesh networks, wireless land area network (WLAN), cellular networks, or the like. A wireless network may be configured to include a system of terminals, gateways, routers, or the like coupled by wireless radio links, or the like, which can move freely, randomly, or organize themselves arbitrarily, such that network topology can change, at times even rapidly.

A wireless network can further employ a plurality of network access technologies, including Wi-Fi, Long Term Evolution (LTE), WLAN, Wireless Router (WR) mesh, or 2nd, 3rd, 4th, 5th generation (2G, 3G, 4G, or 5G) cellular technology, or the like. Network access technologies can allow wide area coverage for devices, such as user devices with varying degrees of mobility, for example.

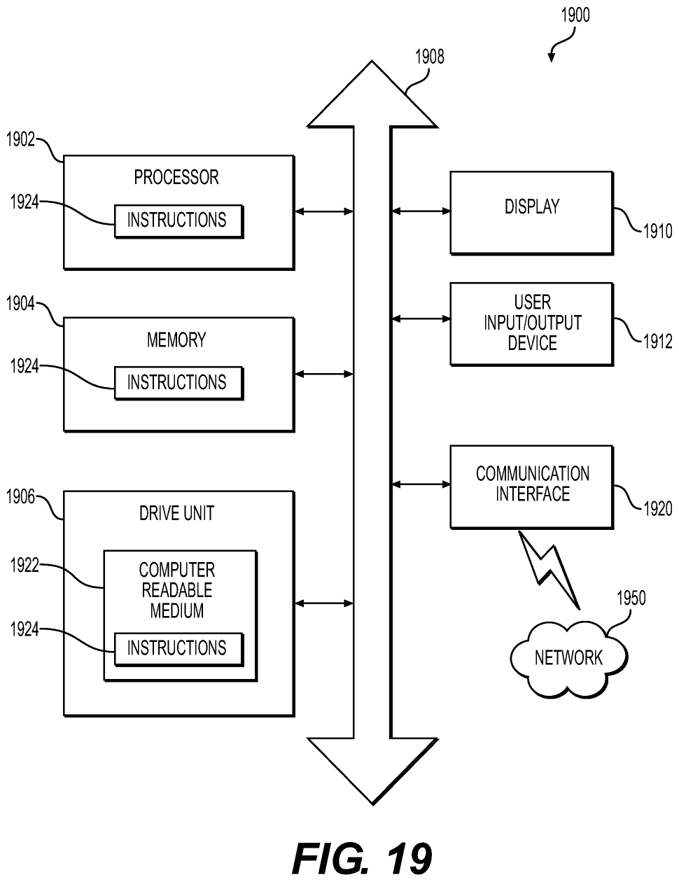

The user device 104 may include any electronic equipment, controlled by a processor (e.g., central processing unit (CPU)), for inputting information or data and displaying a user interface. A computing device or user device can send or receive signals, such as via a wired or wireless network, or can process or store signals, such as in memory as physical memory states. A user device may include, for example: a desktop computer; a mobile computer (e.g., a tablet computer, a laptop computer, or a notebook computer); a smartphone; a wearable computing device (e.g., smart watch); or the like, consistent with the computing devices shown in FIG. 19.

The server device 106 may include a service point which provides, e.g., processing, database, and communication facilities. By way of example, and not limitation, the term “server device” can refer to a single, physical processor with associated communications and data storage and database facilities, or it can refer to a networked or clustered complex of processors, such as an elastic computer cluster, and associated network and storage devices, as well as operating software and one or more database systems and application software that support the services provided by the server. The server device 106, for example, can be a cloud-based server, a cloud-computing platform, or a virtual machine. Server devices 106 can vary widely in configuration or capabilities, but generally a server can include one or more central processing units and memory. A server device 106 can also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

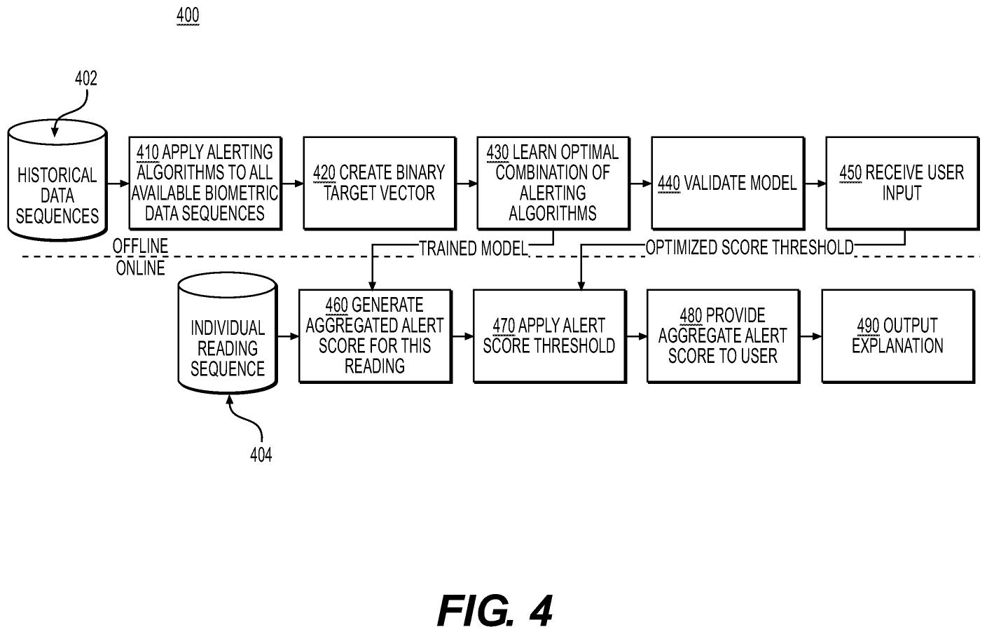

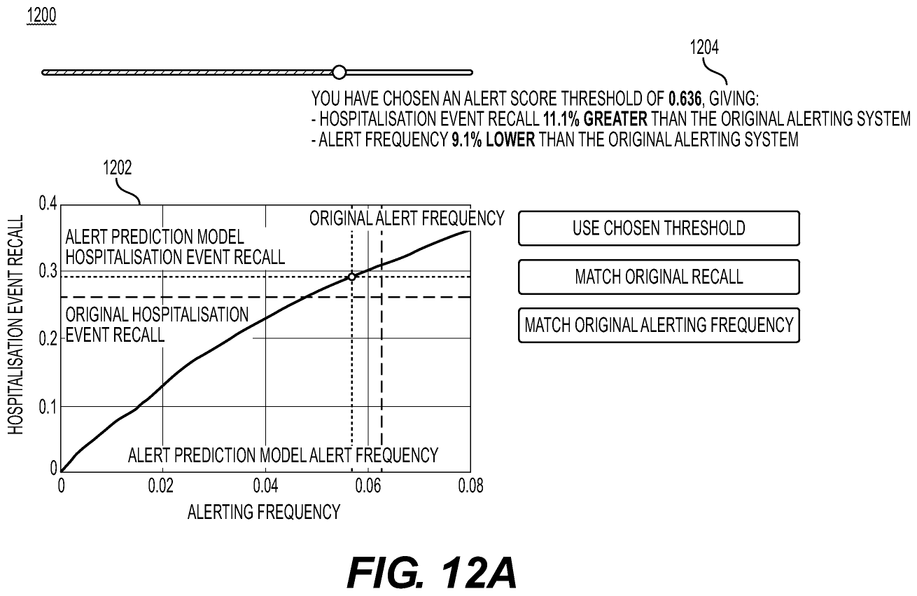

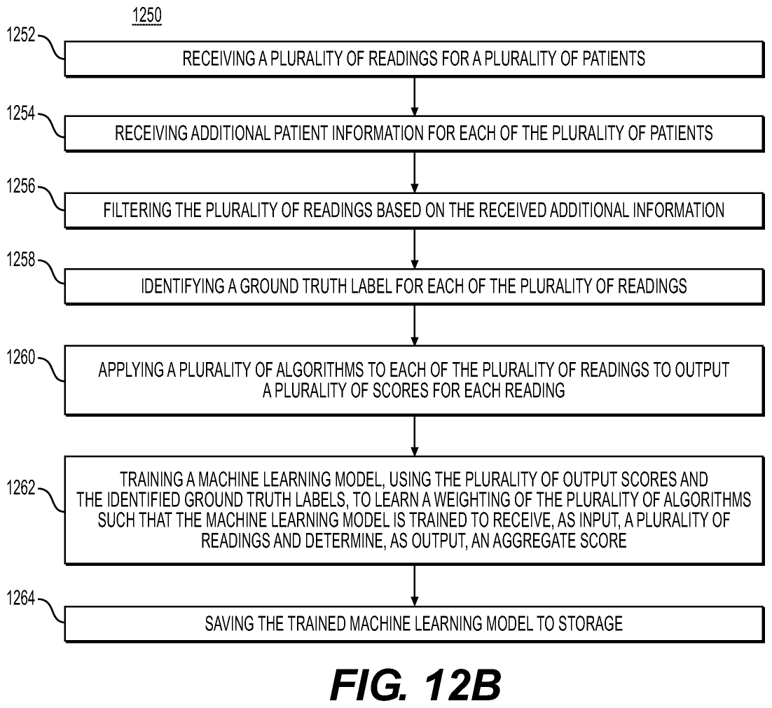

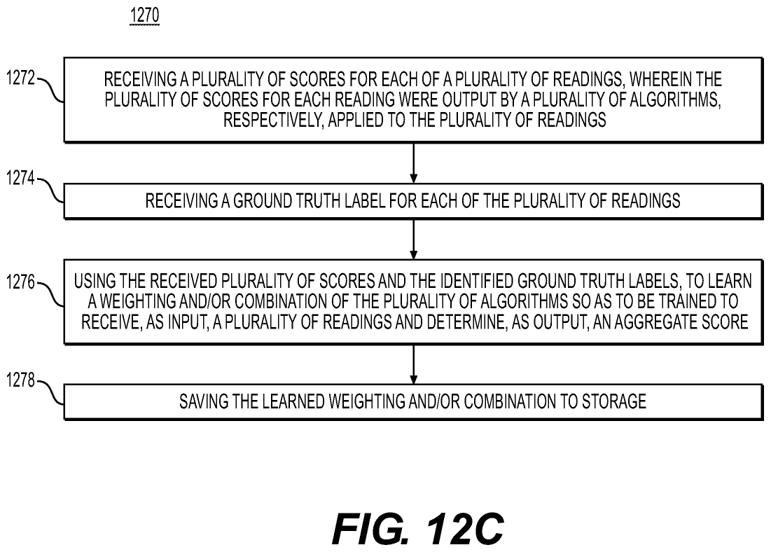

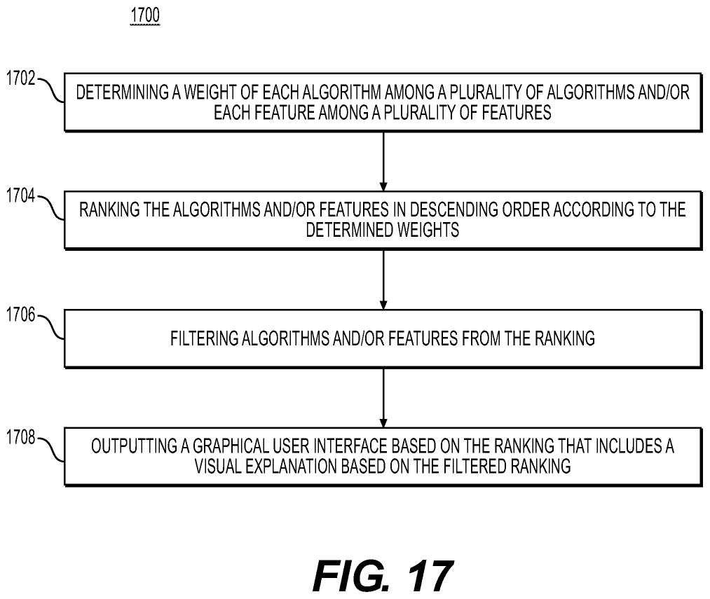

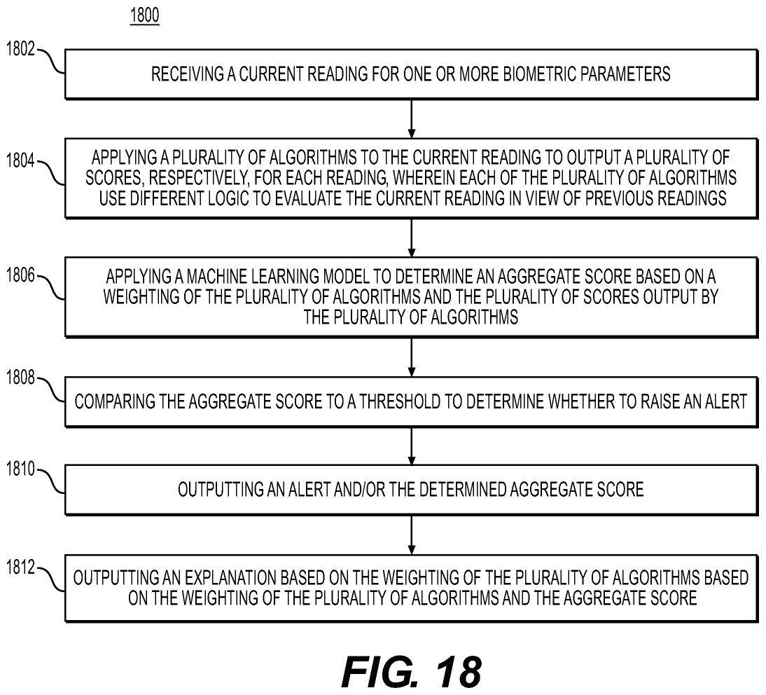

The alerting algorithm balancing platform 108 may include a computing platform hosted on one or more server devices 106. The alerting algorithm balancing platform 108 may provide certain modules, databases, user interfaces, and/or the like for performing certain tasks, such as data processing and/or analysis tasks. For example, the alerting algorithm balancing platform 108 may perform the method illustrated in FIG. 4, the method 1250 illustrated in FIG. 12B, the method 1270 illustrated in FIG. 12C, the method 1700 illustrated in FIG. 17, and/or the method 1800 illustrated in FIG. 18. In some embodiments, a user may use a user device 104 to access one or more user interfaces associated with the alerting algorithm balancing platform 108 to control operations of the alerting algorithm balancing platform 108.

The data store 110 may include one or more non-volatile memory computing devices that may store data in data structure, databases, and/or the like. The data store 110 may include or may be hosted on one or more server devices 106. In some embodiments, the data store 110 may store data related to and/or used for intervention evaluation, output from the alerting algorithm balancing platform 108, and/or the like.

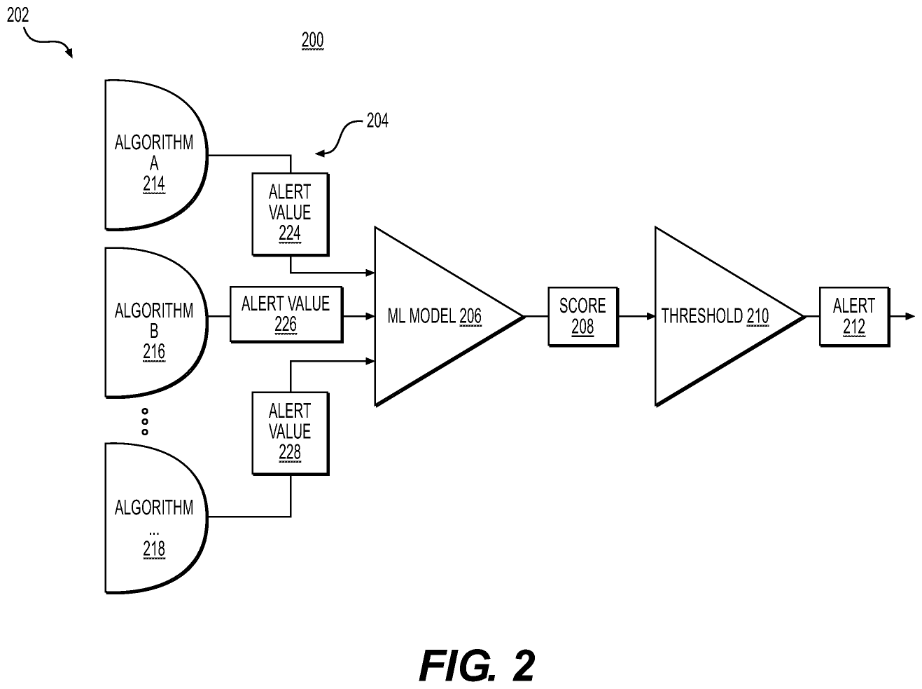

FIG. 2 depicts a flow chart illustrating exemplary processing steps in an exemplary remote patient monitoring (RMS) system 200, according to an exemplary embodiment. The RMS system 200, or at least some portions thereof, may be implemented as the alerting algorithm balancing platform 108 described with reference to FIG. 1. Referring to FIG. 2, the RMS system 200 may include a plurality of algorithms 202 configured to generate a plurality of alert values or scores 204, respectively, and a machine learning model 206 configured to generate a score 208, compare the score 208 to a threshold 210, and output an alert or notification 212 based on the comparison. As used herein, “alert” may mean any notification, prompt, or indication of information and may not necessarily be synonymous with a sounding alarm, blinking light, etc.

The plurality of algorithms 202 may include a first algorithm or “Algorithm A” 214, a second algorithm or “Algorithm B” 216, etc. up to an Nth algorithm 218, and the plurality of alert values or scores 204 may include a first alert value or score 224, a second alert value or score 226, etc., up to an Nth alert value or score 228. The first algorithm 214, the second algorithm 216, and the Nth algorithm 218 may use different logic or calculations and/or be configured to analyze different biometric parameters. Although three algorithms 202 are shown in FIG. 2, aspects disclosed herein are not limited to three algorithms 202 and may include, for example, two, thirty, hundreds, etc. of individual algorithms.

The system 200 may execute the first algorithm 214 to generate the first alert value 224. For example, the first algorithm 214 may use first logic, such as a first statistical process, calculation, or method, to analyze a first biometric parameter for a patient to generate the first alert value 224. The first alert value 224 may be a score (e.g., a probability of a hospitalization event) and/or a binary indication (e.g., 0 or 1) of whether an alert should be raised according to the first algorithm 214.

The second algorithm 216 may use second logic, such as a second statistical process calculation, or method, to analyze the first biometric parameter for the patient to generate the second alert value 226. The second alert value 226 may be a score (e.g., a probability of a hospitalization event) and/or a binary indication (e.g., 0 or 1) of whether an alert should be raised according to the second algorithm 216.

Similarly, the Nth algorithm 218 may use Nth logic, such as an Nth statistical process, calculation, or method, to analyze the Nth biometric parameter for the patient to generate the Nth alert value 228. The Nth alert value 228 may be a score (e.g., a probability of a hospitalization event) and/or a binary indication (e.g., 0 or 1) of whether an alert should be raised according to the Nth algorithm 218.

The first algorithm 214, the second algorithm 216, and the Nth algorithm 218 may also be configured to analyze a second biometric parameter, etc. using their respective first, second, and Nth logics. Alternatively, the plurality of algorithms 202 may include additional algorithms configured to analyze the second biometric parameter and/or other parameters.

The machine learning model 206 may, as an example, be a classification model, but aspects disclosed herein are not limited. The machine learning model 206 may analyze all of the plurality of scores and/or outputs 204 received to produce a score 208, which may be an aggregate score based on a weighting of the plurality of algorithms 202 and/or the plurality of scores 204. For example, the machine learning model 206 may have been trained on prior patient data and events and learned relationships and/or weightings to assign each of the first algorithm 214, the second algorithm 216, and the Nth algorithm 218 and/or each of the first alert value 224, the second alert value 226, and the Nth alert value 228 based on certain situations, certain patient characteristics, etc. to maximize accuracy in determining clinically relevant alerts and/or predicting patient hospitalization. The machine learning model 206 may have been trained to learn certain combinations of situations, patient characteristics, alert values, etc. where a certain algorithm among the plurality of algorithms 202 may produce a false alert or an alert in a situation that is not clinically relevant, and accordingly adjust a weighting.

The machine learning model 206 or another module or processor in system 200 may compare the score 208 to a threshold 210 to determine a final alert value and/or whether to provide an alert 212. The machine learning model 206 and/or another module or processor in system 200 may detect or determine a threshold 210 for a given situation based on a user input of the threshold 210 and/or based on other received information, such as input as to a desired alert frequency, patient data, etc. If the score 208 is above (or alternatively, below, depending on the situation and/or the learned relationships) the threshold 210, the RMS system 200 may output the alert 212 to notify a clinician or other user that the patient needs attention and/or may output the alert 212 that is otherwise indicative of the patient's condition.

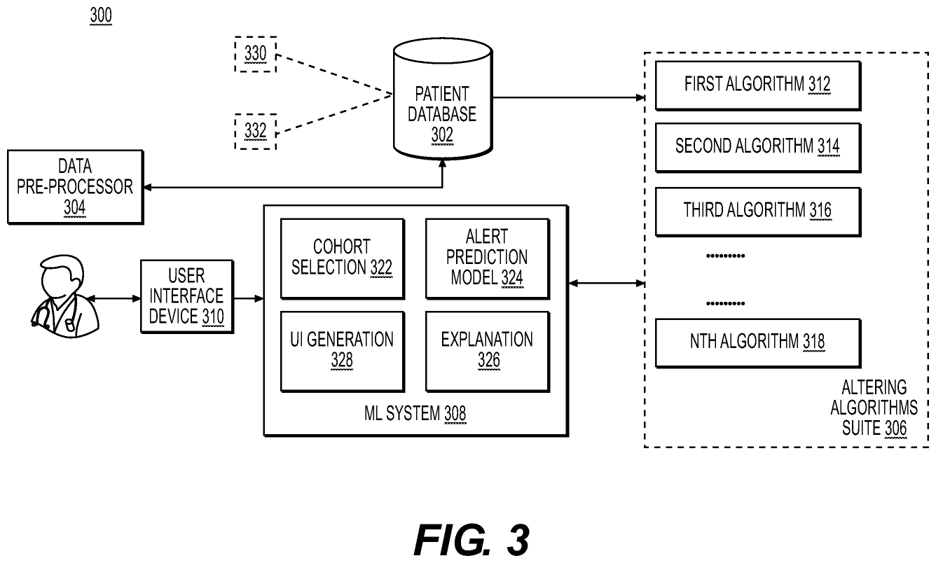

FIG. 3 depicts an exemplary remote patient monitoring (RMS) system 300, according to an exemplary embodiment. The RMS system 300, or at least some portions thereof, may be implemented as the alerting algorithm balancing platform 108 described with reference to FIG. 1 and/or the RMS system 200 of FIG. 2.

The RMS system 300 may include a patient database 302, an alerting algorithms suite 306 including a plurality of algorithms, a machine learning system 308 configured to perform one or more backend processes, and a user interface (UI) 310. The patient database 302 may be implemented and/or include the data store 110 discussed with reference to FIG. 1. The alerting algorithms suite 306 may include the plurality of algorithms 202 discussed with reference to FIG. 2 and/or additional algorithms. The machine learning system 308 (or an alert prediction model 324 thereof) may be implemented as and/or include the machine learning model 206 described with reference to FIG. 2 and/or the alerting algorithm balancing platform 108 described with reference to FIG. 1.

The patient database 302 may include non-sequential or historical data 330 and sequential data or in-patient data 332. The non-sequential data 330 may include patient information such as demographic information (e.g., weight, age, gender, height, body mass index or BMI, etc.), location, comorbidities (e.g., comorbidities at a time of a reading), current or past medications, diagnoses, treatment history or information, care programs a patient is currently enrolled in, physician notes, patient intake data, medical history, recent lab results, previous health events encountered, hospital admissions data, demographic and/or clinical metadata, etc. The sequential data 332 may include readings or other measurements and treatment (e.g., in response to the readings). For example, the sequential data 332 may include blood sugar readings, weight readings, heart rate, heart rate variability, temperature, breathing rate and/or volume, lab work (e.g., from blood readings, such as cholesterol, iron, etc.) and also include hospitalization data, surgery data, etc. The patient database 302 may be connected to and/or in communication with a data pre-processor 304 configured to analyze (e.g., sort and/or classify) the non-sequential data 330 and/or the sequential data 332 in the patient database 302.

The alerting algorithms suite 306 may be connected to and/or in communication with the patient database 302 to receive non-sequential data 330 and/or the sequential data 332. Alternatively or in addition thereto, the alerting algorithms suite 306 may receive information directly from measurement devices, such as a thermometer, scale, heart rate monitor, motion sensor, breathalyzer, etc., and/or input directly from a user through an interface (e.g., a current blood sugar reading and/or objective or subjective evaluations by a clinician).

The alerting algorithms suite 306 may include a plurality of algorithms, each using different logic such as standard deviation, trend detection, recent min/max, regression, clustering, N days change, interquartile, percentile rule, variability rule, alert re-prioritization, etc. The alerting algorithms suite 306 may include multiple variations (e.g., dozens or 30 variations) of an algorithm. For example, the alerting algorithms suite 306 may include dozens of algorithms that use standard deviation (e.g., based on different data sets, different parameters, etc.), dozens of algorithms that use regression or clustering techniques, dozens of algorithms that use trend detection, etc. This list of algorithms is not exhaustive.

The alerting algorithms suite 306 may include a first algorithm 312, a second algorithm 314, a third algorithm 316, etc. up to an Nth algorithm 318. As an example, the first algorithm 312 may be a standard-deviation based algorithm that analyzes all received data (e.g., non-sequential data 330 and/or sequential data 332) by calculating, for example, one or more standard deviations for one or more biometric parameters. The second algorithm 314 may be a standard-deviation based algorithm that analyzes data received after hospitalization (e.g., non-sequential data 330 and/or sequential data 332 measured after hospitalization) by calculating, for example, one or more standard deviations for one or more biometric parameters measured after hospitalization. The third algorithm 316 may be a regression algorithm that analyzes all received data (e.g., non-sequential data 330 and/or sequential data 332) using regression techniques. The Nth algorithm 318 be a clustering algorithm that analyzes all non-sequential data 330 and/or sequential data 332 using clustering techniques. As previously explained, these implementations of the first algorithm 312, the second algorithm 314, the third algorithm 316, etc. up to the Nth algorithm 318 are exemplary (i.e., are merely a non-exhaustive list of examples) and may not describe all types of algorithms or logic in the alerting algorithms suite 306.

The machine learning system 308 may include an alert prediction model 324, which may execute at least some of the plurality of algorithms in the alerting algorithms suite 306 to determine one or more alert values or scores. The machine learning system 308 may be configured to perform other processes, such as cohort selection 322 (using, for example, a cohort selector or selection model), one or more explanations of the findings of the alerting algorithms suite 306 (using, for example, an explainer model and/or the alert prediction model 324), and user interface generation (using, for example, a user interface generator or model).

As described with reference to the machine learning model 206 of FIG. 2., the alert prediction model 324 may be a model trained to balance the plurality of algorithms in the alerting algorithms suite 306 and/or their resulting alerts or scores to determine whether to issue a final alert or notification. The machine learning system 308 may analyze the resulting alerts or scores, in addition to the learned weights by the alert prediction model 324, to analyze a patient condition and/or rank the alerts or scores. For example, algorithms that were given more weight by the alert prediction model 324 may have their associated alerts and/or scores ranked higher, and the machine learning system 308 may use the ranking and/or relative value of scores, etc. to provide an explanation 326 as to the patient's condition. The machine learning system 308 may provide an explanation for all situations and/or be prompted to generate an explanation.

The machine learning system 308 may, via user interface generation 328, determine a user interface (e.g., graphical user interface, dashboard, etc.) that includes the alerts or scores of the alerting algorithms suite 306, any final alert determined by the machine learning system 308, and any explanations 326 determined. For example, the machine learning system 308 may determine a dashboard showing text that explains a patient's condition, graphs showing trends and warnings, the ranking of alerting algorithms and/or their resulting alerts or scores, etc. The determined user interface may be output on a user interface device 310 (e.g., display, hospital monitor, mobile device, printer, etc.) so that a user (e.g., clinician or practitioner) may review the determinations of the machine learning system 308.

The RMS system 300 may thus, via machine learning system 308 and alerting algorithms suite 306, evaluate each of a plurality of alerting algorithms through learning an optimal weighting among them and/or their resulting alerts or scores (e.g., probabilities) to estimate a need of raising an alert to a clinician, patient, etc. ahead of a hospitalization event. The optimal weighting may be configured to reduce alert frequency and increase accuracy and/or hospitalization event recall. The alerting algorithms suite 306 may process readings from multiple biometric types and learn weights for each type.

The RMS system 300 may provide a highly personalized, automated, and explainable alerting system that reduces clinicians' alert fatigue by detecting biometric patterns highly associated with negative health events. In addition, the RMS system 300 may build an explainable alerting solution using an alerting algorithms suite 306 having different algorithms, some of which may be relatively simple, information from patient database 302 and/or other received information. The machine learning system 308 may determine explanations and/or decide whether an alert should be produced for a patient (e.g., via user interface device 310 or other output device) considering their recent biometric readings and medical details.

The RMS system 300 may adjust a prediction process and/or alert determination process based on a user's feedback. For example, through the user interface device 310, a clinician may input a desired alerting frequency, which may be considered by machine learning system 308 to determine whether to provide an alert (e.g., using the user interface device 310 and/or another output device, such as a pager, cell phone, computer, etc.)

The RMS system 300 may provide transparency by providing explanations 326 and/or by generating a user interface 328 that shows a reasoning process of the machine learning model 308 (e.g., by showing a ranking of each individual parameter, factor, score, alert value, etc. used in the determination of whether to provide an alert) so that a clinician may make more informed decisions. In some examples, the RMS system 300 may provide an alert only if a patient is at risk of a relevant adverse health event.

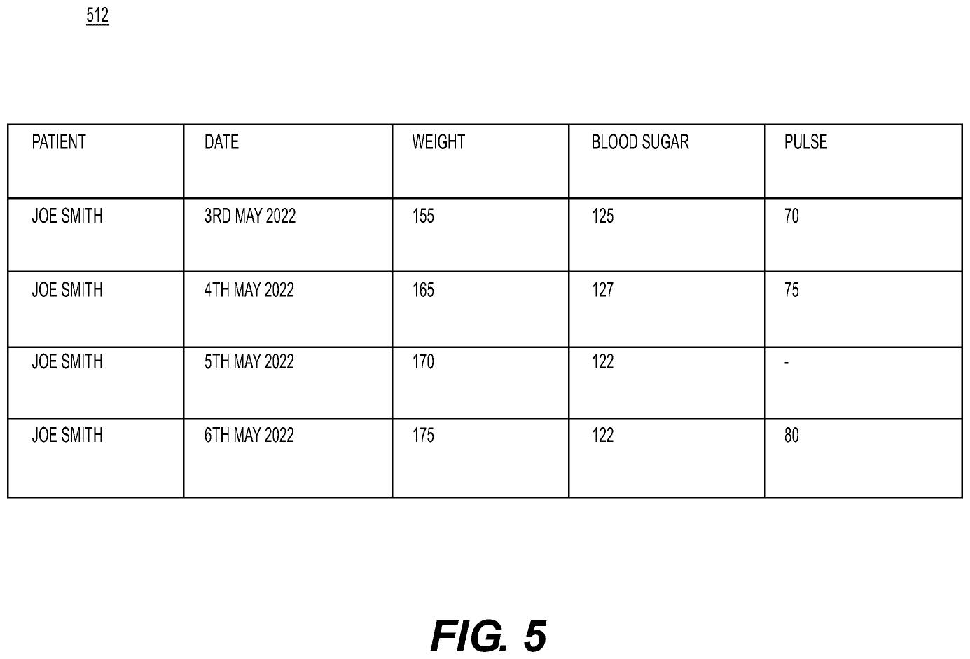

FIG. 4 outlines a method 400 of training and using an RMS system, according to an exemplary embodiment, including offline and online steps or tasks. The steps outlined in FIG. 4 will be explained in more detail with reference to FIGS. 5-17.

Referring to FIG. 4, to build a model, the method 400 may include, at step 410, applying a plurality of alerting algorithms to all available biometric data sequences. The plurality of alerting algorithms may receive historical data sequences 402 for a plurality of historical patients. The method 400 may include, at step 420, creating or determining a binary target vector based on the outputs of the alerting algorithms at step 410. The method 400 may include, at step 430, learning an optimal combination and/or weighting of the plurality of alerting algorithms, by training a machine learning model. The optimal combination may be a combination that reduces alert frequency as much as possible while maintaining a high accuracy or hospitalization event recall (or increases an accuracy or hospitalization event recall as much as possible). The method 400 may include, at step 440, validating the learned optimal combination and/or weighting, or validating the machine learning model. The method 400 may include, at step 450, receiving input from one or more users (e.g., clinician or stakeholder) regarding trade-offs or alerting frequency.

In using the model to evaluate an individual reading sequence 404, the method 400 may include, at step 460, generating an aggregate alert score. Step 460 may apply the plurality of algorithms based on the learned optimal combinations and/or weightings determined at step 430 (e.g., by applying a machine learning model trained at step 430). The method 400 may include, at step 470, applying an alert score threshold, which may include comparing the aggregate alert score generated at step 460 with the alert score threshold. The alert score threshold may be an optimized score threshold based on the input received from the one or more users at step 450. The method 400 may include, at step 480, providing the aggregated alert score to a user and/or providing an alert or notification. The method 400 may include, at step 490, outputting an explanation and/or reasoning for an alert or notification.

Applying a plurality of alerting algorithms to all available biometric data sequences at step 410 may include applying a suite of alerting algorithms (e.g., alerting algorithms suite 306 described with reference to FIG. 3) to biometric reading data for all patients and outputting one alerting vector per algorithm. The biometric reading data may include one or more readings recorded for a patient on a regular basis or based on a schedule. These readings may be from a variety of biometric types, including heart rate or pulse, blood pressure systolic, blood pressure diastolic, weight or blood sugar, temperature, etc. FIG. 5 shows an example of biometric reading data 512 for an individual patient recorded over several days.

As previously described with reference to FIG. 3, the suite of alerting algorithms may include a plurality of algorithms. Each algorithm may use certain logic (e.g., standard deviation, regression, clustering, trend, recent min/max, etc.) to detect whether a current reading for a given patient is abnormal when compared to a history of readings for that same patient. If the algorithm determines that the current reading is abnormal, then it may raise an individual alert, may generate an alert score or value (e.g., proportional to an extent of an abnormality and/or a probability of hospitalization based on the reading), and/or output a binary indication of whether an alert should be raised (e.g., 0 or 1).

Each individual algorithm among the plurality of algorithms may implement different logic to analyze the patient's data and flag a particular pattern. A logic behind a single algorithm may be based on a deviation from one or more bounds calculated from a distribution of past reading values and/or based on a trend derived from the past reading values. In some examples, each alerting algorithm may yield a binary output, such as “Alert” or “No Alert,” for every reading of every patient.

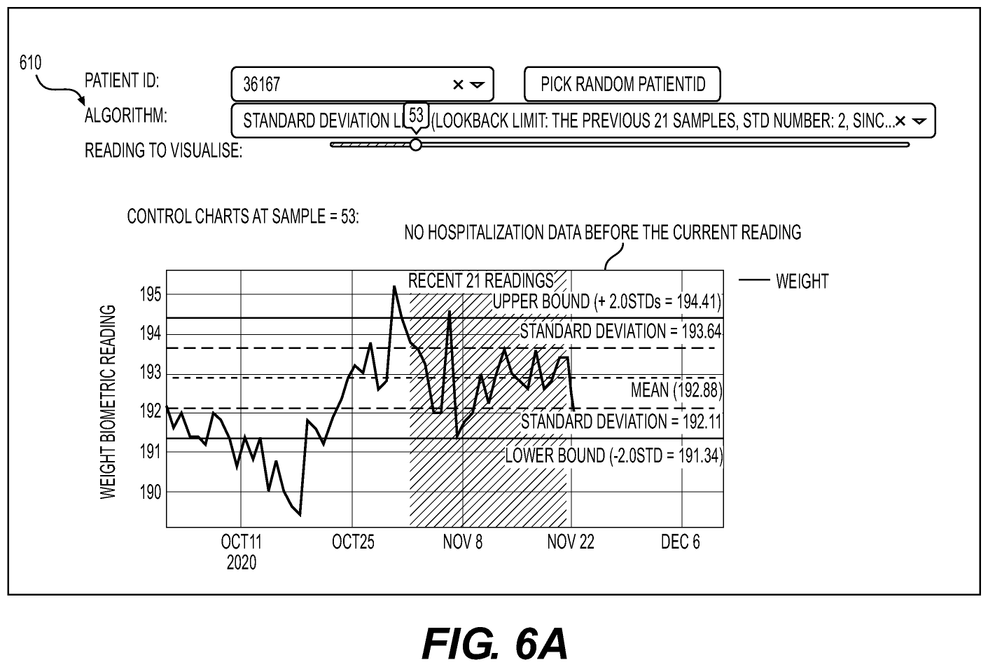

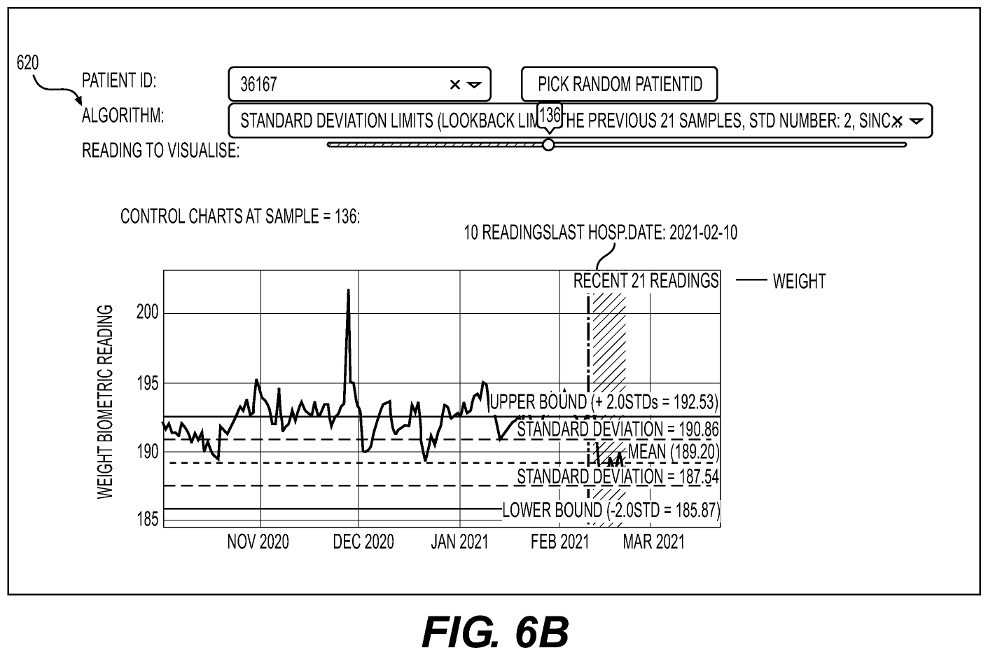

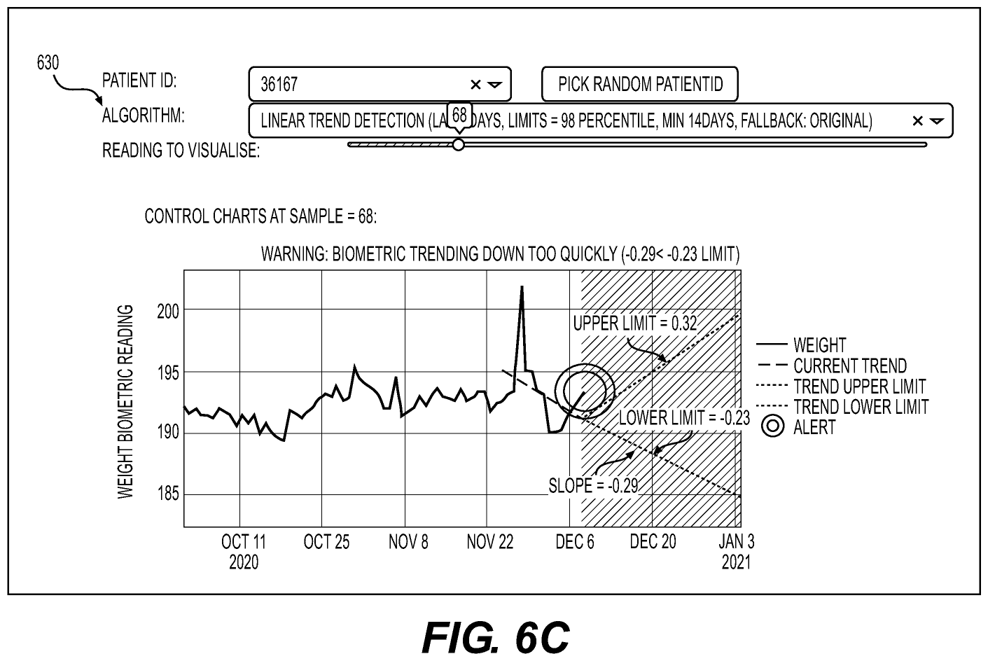

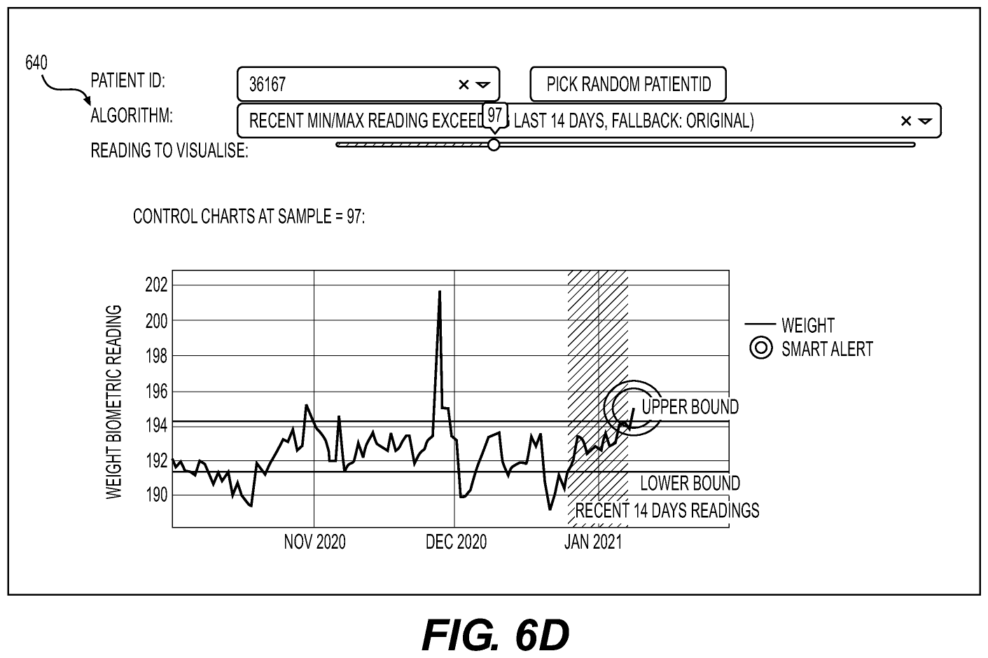

FIGS. 6A-6C are exemplary displays that analyze biometric reading data using respective alerting algorithms. Each display, or graphical user interface, may be designed to explain the results of the corresponding alerting algorithm using a graph illustrating relevant parameters and variables. These displays may be presented to a user (e.g., a patient, a health care provider, etc.), helping the user in observing and understanding the results between different alerting algorithms. Referring to FIG. 6A, a first example of an alerting algorithm (e.g., first algorithm) 610 may use a statistical process control methodology and/or standard deviation based logic. The first algorithm 610 may calculate a mean and a standard variation based on all historical biometric readings of a patient, and determine to raise an alert or notification if a current reading value is outside 2 standard deviations from the mean. Different variations of this logic may be implemented as separate alerting algorithms. For example, some variations may use a different count of standard deviation (other than 2) to determine upper and lower bounds or may focus on specific parts of past readings data instead of using all data to calculate the bounds, such as using only the readings since the last hospitalization event or using only the most recent readings (e.g., from the last two weeks). For Example, FIG. 6A shows an example where the first algorithm 610 used the last 21 readings to generate alerts, while FIG. 6B shows a second example of an alerting algorithm (e.g., a second algorithm) 620 that uses only the readings after a recent hospitalization event to generate alerts. FIGS. 6A and 6B each show a display or user interface that shows a trend or reading line or chart of a biometric parameter (e.g., weight) and values determined by their respective algorithms (e.g., values for mean and standard deviation, upper and lower bounds or minimums or maximums of the readings, etc.). The displays may indicate, on the reading line, past readings that triggered alerts and/or various levels of alerts (e.g., a medium alert and/or a high alert). In FIGS. 6A through 6D, the alert may be based on one algorithm alone (e.g., the first algorithm 610 in FIG. 6A or the second algorithm 620 in FIG. 6B) and/or on a family of algorithms alone and not in combination with multiple algorithms that significantly use different logic.

Referring to FIG. 6C, a third example of an alerting algorithm (e.g., third algorithm) 630 may use logic based on trends. For example, the third algorithm 620 may estimate a linear trend of recent readings (e.g., readings taken in the previous 14 days) and raise an alert for a current biometric reading if a trend value is above a limit or threshold (e.g., a user-tailored limit, a predetermined limit, a learned limit, etc.). In the example of FIG. 6C, a display may show a trend or reading line of a biometric parameter (e.g., weight) based on previous readings and a current reading of the biometric parameter. The display may indicate, on the reading line, past readings that triggered alerts and/or various levels of alerts (e.g., a medium alert and/or a high alert).

The display may also display a recent trend line which may indicate a current trend determined by the third algorithm 630, a trend upper limit line, and a trend lower limit line. The trend upper limit line may indicate values that trigger an alert if the current trend line goes above those values, and the trend lower limit line may indicate values that trigger an alert if the current line goes below those value. The trend upper limit and lower limit lines may be determined by the third algorithm 630 based on past trends and/or slope values (e.g., the 1^{st} and 99^{th} percentile of all past trend data).

The third algorithm 630 may determine that a biometric parameter (e.g., weight) is trending downward at a rate that is greater than a predetermined threshold and/or at a rate that is faster than an average rate, and may display an alert or warning (for example, “WARNING: BIOMETRIC TRENDING DOWN TOO QUICKLY”). The third algorithm 630 may be a “smart algorithm” or an algorithm that is not based on a simple threshold. The third algorithm 639 may determine an alert for a current reading (e.g., shown by an area or circle on the display).

Referring to FIG. 6D, a fourth example of an alerting algorithm (e.g., fourth algorithm) 640 may use logic based on recent minimums and/or maximums in data. For example, the fourth algorithm 640 may use maximum and minimum readings of recent readings (e.g., readings taken in the previous 14 days) and set these values as upper and lower bounds, respectively. The fourth algorithm 640 may raise an alert for a current biometric reading if the current reading exceeds either of these set bounds.

Referring back to FIG. 4, the historical data sequences 402 may include historical patient hospitalization data. For example, the historical data sequences 402 may include hospitalization dates, outcome data, and/or diagnosis data (e.g., associated primary diagnosis codes (ICD10)). The various diagnosis information may be grouped in categories and/or ranked. For example, the primary diagnosis codes may be grouped into Hierarchical Condition Categories (HCC) as defined by the Centre for Medicare and Medicaid Services (CMS).

The historical patient hospitalization data may be used to build a model that is specific to certain diagnoses and/or certain patient characteristics. To build a model that provides alerts for a pre-defined set of conditions, the hospitalization events in the historical data sequences 402 may be filtered by a specific HCC category. For example, HCC85 may be a category grouping for ICD10 codes related to heart failure. To build a model that provides alerts in advance of hospitalization for heart failure, hospitalization events with a primary diagnosis code in this category may be pulled from the historical data sequences 402 to be fed to the alerting algorithms suite.

Aspects disclosed herein may use verified medical events, such as hospitalization admission events, to approximate ground truth labels in training the model. For a biometric reading for a given patient, ground truth may be defined by whether an alert should be raised, or whether the patient will be hospitalized within a given time window. Ground truth labels may be assigned by checking whether a biometric reading lies within a predetermined period time of a medical event, such as an admission window of a hospitalization event. An admission window for a given hospitalization event may be a period of time before the hospitalization event.

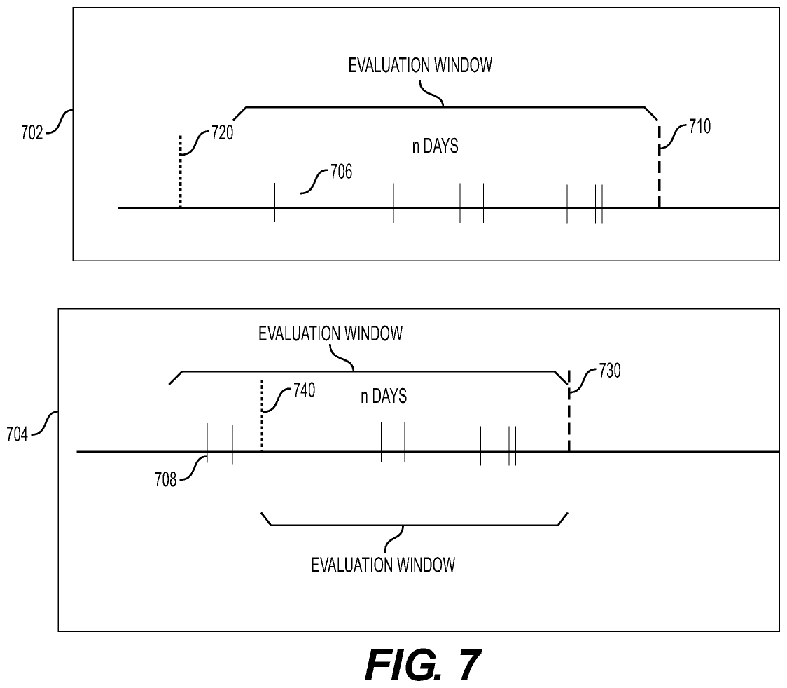

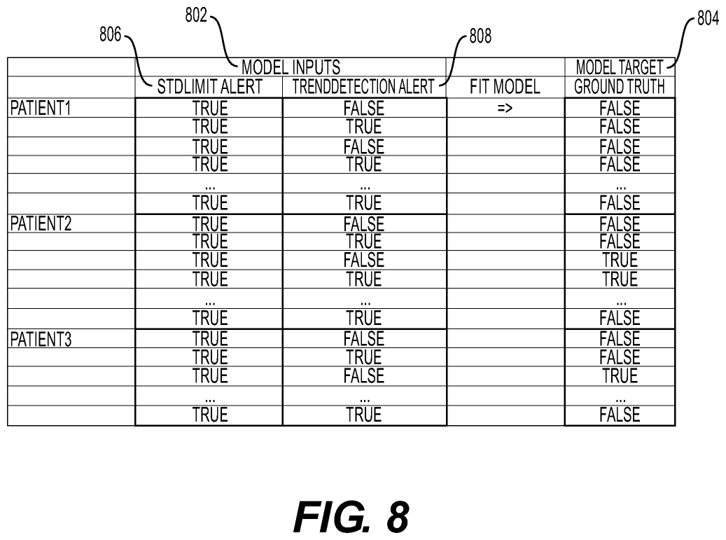

Referring to FIG. 7, a default admission or evaluation window for a given hospitalization event may be expressed as “n days” before an admission date of the hospitalization event. The number of days n may be determined based on a condition that led to the event. For example, the number of days n may be between 7 and 14 days. In some examples, a model (e.g., a machine learning model) or machine learning system may be used to learn a value of “n” for certain types of events or patients (e.g., heart failure events).

If no other hospitalization or admission date occurred during the last n days before a current admission date 710, then a calculated admission or evaluation window may be the same as the default admission window of n days, as shown in a first scenario 702. The admission window may begin on a date that is n days before the current admission date 710 and end at the current admission date 710, and may be expressed as [current_admission_date-n days, current_admission_date].

Any prior admission, such as previous admission date 720, that occurred before n days is not considered in the calculation of the admission date.

If, however, a previous hospitalization occurred on a previous admission date 740 that is within the last n days of a current admission date 730, then the calculated admission window may be shortened from the default admission window to a period between the previous hospitalization date 740 and the current admission date 730, as shown in a second scenario 704. This admission window for the admission date 730 may begin on the previous admission date 740 and end on the current admission date 730, and may be expressed as [previous_admission_date, current_admission_date].

If a reading lies within the calculated admission window, such as first reading 706, then it may be assigned a ground truth label of “True” or 1. If the reading lies outside the calculated admission window, such as reading 708, the reading may be assigned a ground truth label of “False” or 0. In the second scenario of FIG. 7, although reading 708 may lie outside the calculated admission window for the current admission date 730, reading 708 may lie inside of a calculated admission window date for the previous admission date 740. If the previous admission date 740 has same or similar characteristics as the current admission date 730 that the model may be trained to target (e.g., heart failure), the reading 708 may, in this case, be assigned a ground truth label of “True” or 1 if the reading 708 lie within a calculated admission window of previous admission date 740.

Referring to FIGS. 4 and 8, applying the alerting algorithms at step 410 may include determining the ground truth label of readings, and, for those readings labeled as true or within the admission window, determining whether to raise an alert. Creating a binary target vector at step 420 may include outputting a ground truth binary vector or a ground truth matrix. Each of the entries in this ground truth binary vector may indicate a ground truth label for a single reading of a single patient.

Although FIG. 7 illustrates using hospitalization admission data for ground truth, in some examples, ground truth labels may be assigned based on other verified medical events, such as other trauma that did not result in hospitalization, where such data may be available. For example, if a patient was not admitted to the hospital but suffered from a traumatic event before they could be admitted (e.g., death), then a date of this trauma, if available in the historical data sequences 402 and/or patient database 302, may be used where there may not be a hospitalization admission date.

Other verifiable trauma (e.g., severe sickness where the patient may have been treated by a physician or other medical professional but not admitted to a hospital) could also be used in some models, depending on what the model is intended to predict. In some examples, the system may continuously collect data from certain patients being monitored outside of a hospital (e.g., via heart rate monitors or other portable sensors), and certain less severe but still significant events (e.g., fainting, high heart rate, etc.) may be used for other types of alerts. In such an example, an “admission window” may be a number of minutes, hours, etc. before the event, and another reading occurring during the window may be assigned a ground truth label of true for a model aimed to predict alerts for those types of events.

FIG. 8 illustrates a model inputs matrix 802 including readings data and a model target or ground truth matrix 804 generated based on the ground truth data. Here, the model inputs matrix 802 and the model target matrix 804 include values for three patients, but aspects disclosed herein may include evaluating data for more than three patients (e.g., dozens, hundreds, thousands, etc.). Each row in the model inputs matrix 802 and the model ground truth target matrix 804 corresponds to one biometric reading. The model inputs matrix 802 may include all biometric readings for a patient, all biometric readings during a certain period of time, or all biometric reading that was flagged by at least one algorithm as raising an alert.

The model inputs matrix 802 may show results of the plurality of alerting algorithms, which may include a first algorithm 806 and a second algorithm 808. The results may indicate whether an individual algorithm determined that an alert should be raised. For example, “True” may indicate that, for that reading, an individual algorithm determined that an alert should be raised, while “False” may indicate that, for that reading, an individual algorithm determined that an alert should not be raised.

Aspects disclosed herein may include evaluating data with more than two algorithms (e.g., dozens, hundreds, etc.). In the example of FIG. 8, the model inputs matrix 802 implements the first algorithm 806 as a standard deviation algorithm and the second algorithm 808 as a trend detection algorithm. As exemplified in the model inputs matrix 802, the first algorithm 806 may determine a different label than the second algorithm 806.

The model target matrix 804 may indicate, for each reading, a ground truth label determined based on hospitalization events, as discussed with reference to FIG. 7. The model target matrix 804 may be used as labels for training the machine learning model using the corresponding inputs from the model inputs matrix 802.

Learning optimal combinations and/or weightings of algorithms at step 430 may include feeding the alert results of applying the alerting algorithms suite and the binary target vector to a machine learning model. The machine learning model may take, as input, the outputs of the suite of alerting algorithms applied at step 410 (which may be reflected in a model inputs matrix 802) and the ground truth labels assigned and/or the binary target vector created at step 420 (which may be reflected in a model target matrix 804). The machine learning model may learn patterns and be trained to take, as input, outputs of a suite of alerting algorithms applied to individual reading data (for example, individual reading sequence 404), predict, for each reading, whether a reading result will result in hospitalization, and output an indication of whether an alert should be raised. The output of the machine learning model may be configured to indicate a likelihood of an alert being raised due to hospitalization at a horizon of n days.

The machine learning model, may, for example, be any classifier configured to produce continuous probability and/or score outputs (e.g., logistic regression, random forest, xgboost, etc.) and may learn how much each individual algorithm in the alerting algorithms suite contributes to a prediction of alerts related to a specific hospitalization event. The machine learning model may, for example, use deep neural networks to learn weights of the contributing individual algorithms and/or use more complex neural network feature extractors (CNNs, RNNs, feature crosses, etc.) and custom loss functions, enabling further tailoring of a response of the machine learning model to particular types of events.

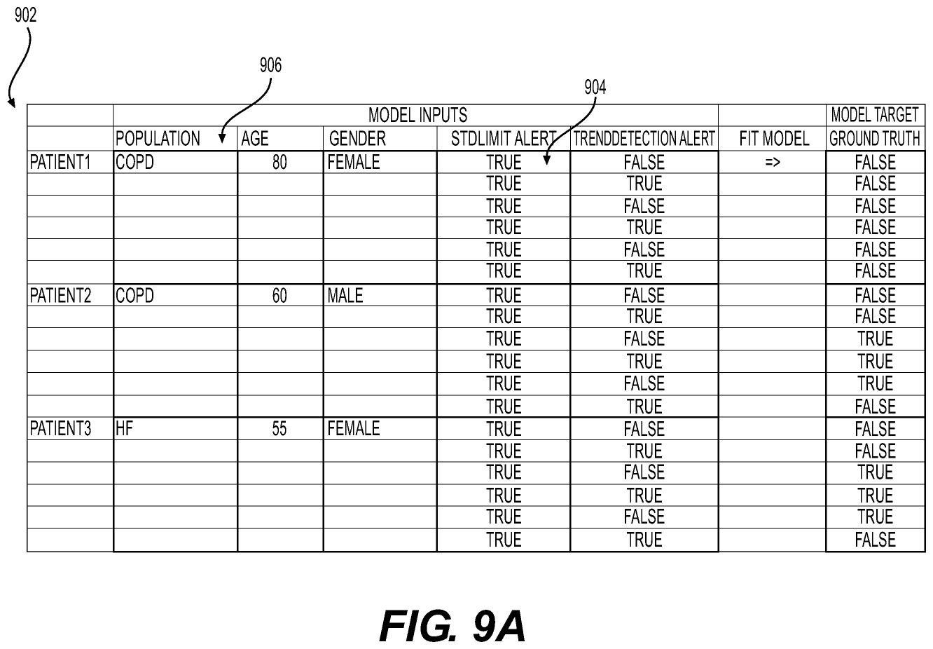

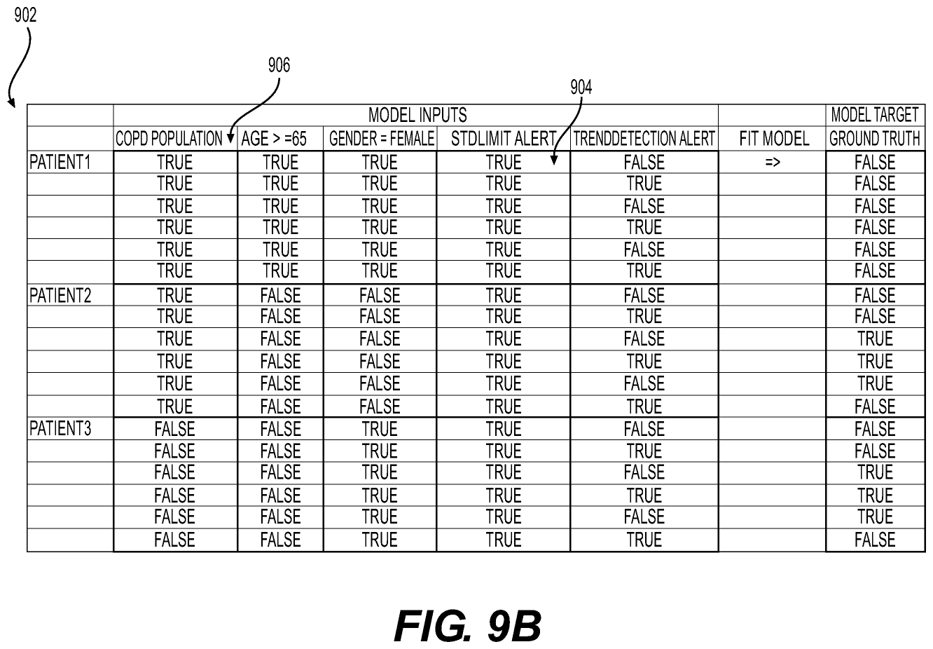

Referring to FIGS. 4 and 9A-9B, in addition to the outputs of the alerting algorithms suite (e.g., binary outputs 904 such as True or False exemplified in the model inputs matrix 902 of FIG. 9), additional patient information 906 may be input into the machine learning model for training at step 430 and also in a use case (e.g., at step 460). For example, the machine learning model may take, as additional input, any demographic and/or clinical input features that are deemed relevant to predicting the hospitalization risk. Although FIGS. 9A-9B show that the additional patient information 906 may include population, age, and gender, aspects disclosed herein are not limited to those characteristics. For example, additional patient information 906 may include weight, age, gender, height, body mass index or BMI, location, comorbidities (e.g., comorbidities at a time of a reading), current or past medications, diagnoses, treatment history or information, care programs a patient is currently enrolled in, physician notes, patient intake data, medical history, recent lab results, previous health events encountered, demographic and/or clinical metadata, etc.

In addition to patient information, where multiple biometric parameters are being fed as input, the information may indicate which readings correspond to which biometric parameters (e.g., heart rate, blood sugar, etc.), and the machine learning model may learn an optimal weighting and/or combinations of biometric parameters. In some examples, certain algorithms may be configured for certain biometric parameters, which may be considered in learning an optimal weighting and/or combinations of biometric parameters and/or the algorithms.

FIG. 9B shows how the additional patient information 906 may be organized to have binary indications. Including this additional patient information 906 may enable the machine learning model to condition its response to certain patient characteristics of an individual for whom the prediction may be being made, hence improving a predictive performance for that individual. In the example of FIG. 9B, the machine learning model may be targeted for female patients over the age of 65 and who have chronic obstructive pulmonary disease (COPD). This additional patient information 906 may enhance interpretability of the machine learning model.

Validating at step 440 may include feeding, to the machine learning model, a validation data set that was not used for training to confirm the learned optimal combinations and/or weightings at step 430. The validation data set may include a model inputs matrix, and the outputs of the machine learning model may be compared to ground truth data and/or a model targets matrix that was not input. The machine learning model may compute an output score (e.g., an alert value or score), which may be compared to a threshold to determine whether an alert should be raised.

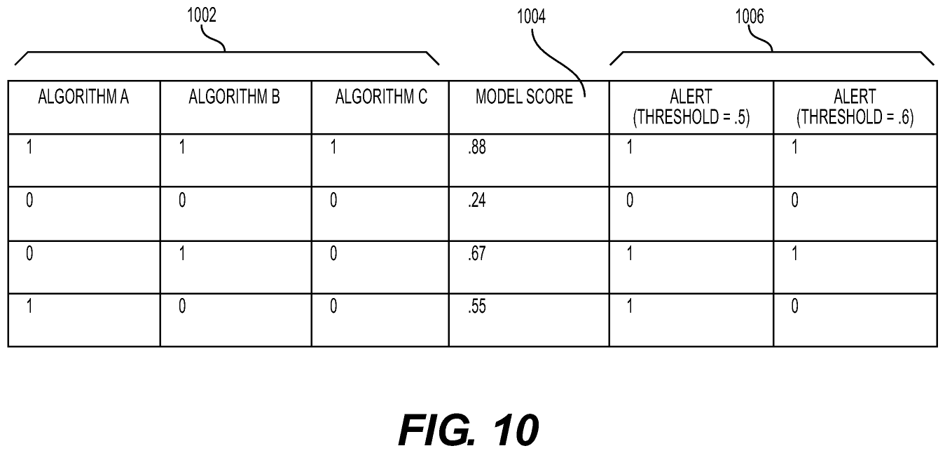

FIG. 10 shows an example of validation results compared to a threshold of 0.5 and a threshold of 0.6. In FIG. 10, each algorithm of a plurality of algorithms 1002 (e.g., Algorithm A, Algorithm B, and Algorithm C) may output an indication of whether an alert should be raised based on a reading. In FIG. 10, each row may represent a reading for a biometric parameters. The machine learning model may determine, for each reading and based on each indication by each of the plurality of algorithms 1002, a model score 1004. This model score 1004 may indicate a probability of hospitalization based on the reading. The model score 1004 may be compared to one or more thresholds 1006, and indicate using a value (e.g., using 1 for “Yes” and 0 for “No) whether the model score 1004 is above the threshold 1006. As shown in FIG. 10, for a model score of 0.55, where the threshold is 0.5, the value is 1 to indicate “Yes,” (or that the model score 904 is at or above the threshold) and where the threshold is 0.6, the value is 0 to indicate “No” (or that the model score 1004 is below the threshold).

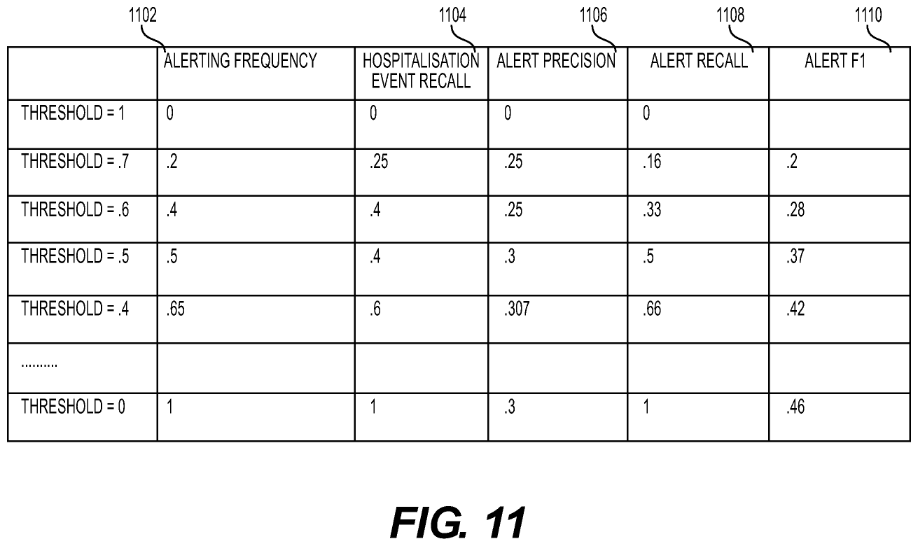

Referring to FIG. 11, different model performance metrics may be used to evaluate the trained machine learning model. For example, as shown in FIG. 11, alerting frequency 1102, hospitalization event recall 1104, alert precision 1106, alert recall 1108, alert F1-score 1108, etc. may be used to evaluate the trained machine learning model, such as by comparing the outputs (e.g., model score 1004 in FIG. 10) to the ground truth and/or hospitalization data. These metrics may be organized by various threshold values to help assess, determine, and/or tune a threshold.

Alerting frequency 1102 may indicate how often and/or a percentage of readings for which the machine learning system raised an alert. For example, as shown in FIG. 11, where the threshold was 0, the machine learning system raised an alert for every reading, as indicated by a value of 1, which may represent 100%. Where the threshold was 1, the machine learning system did not raise any alerts. The hospitalization event recall 1104 may indicate a percentage of hospitalization events that the machine learning model accurately predicted and/or pre-empted.

Alert precision 1106, alert recall 1108, and alert F1 1110 may refer respectively to a precision, a recall, and an F1 score based on true positive counts, false positive counts, true negative counts, false positive counts, threshold value, the model output scores, and the ground truth labels. True positive counts may reflect a number of readings assigned a ground truth label of true based on hospitalization events and where the machine learning model predicted would result in hospitalization. False positive counts may reflect a number of readings that had a ground truth label of false but where the machine learning model predicted would result in hospitalization. True negative counts may reflect a number of readings assigned a ground truth label of false and where the machine learning model did not predict would result in hospitalization. False negative counts may reflect a number of readings assigned a ground truth label of true but where the machine learning model did not predict would result in hospitalization.

For example, alert precision 1106 may indicate or be based on a fraction of true positive counts among all readings the machine learning model indicated would result in hospitalization. Alert precision 1106 may be the true positive counts divided by a sum of the true positive counts and false positive counts. However, aspects disclosed herein are not limited to a formula used for alert precision 1106.

Alert recall 1108 may indicate or be based on a fraction of readings that the machine predicted would result in hospitalization among all readings that were assigned a ground truth label of true. For example, alert recall 1108 may be a sum of true positives and false positives divided by a sum of true positives and false negatives. However, aspects disclosed herein are not limited to a formula used for alert recall 1108. As another example, alert recall 1108 may be a number of true positives divided by a sum of true positives and false negatives.

The Alert F1 1110 may be based on the alert precision 1106 and/or the alert recall 1108. Aspects disclosed herein are not limited to a formula used for the Alert F1 1110.

A curve or graphical representation may be depicted of the model performance metrics as a threshold is varied between a maximum value (e.g., 1) and a minimum value (e.g., 0) based on the validation subset. This curve may be presented to a user (e.g. clinician, institution, or stakeholder) who can decide on an optimal threshold value using their domain knowledge and observing an impact of different threshold values on model performance. For example, the clinician may decide that a lower alerting frequency may be desirable based on understaffing, and pick a threshold that still has an acceptable hospitalization event recall 1104 or other acceptable metrics. In some examples, the machine learning model may determine or tune a threshold based on other inputs. For example, the machine learning model may take, as input, staff assignments, hospital scheduling, and admission rate, and adjust a threshold accordingly based on a workable alerting frequency in view of, for example, a ratio of staff members to admitted patients.

The selected threshold may apply to all biometric parameters, as the machine learning system may output an aggregate score based on a probability, and the threshold may reflect a probability. Alternatively or in addition thereto, the clinician may select a different threshold for each type of biometric parameter (e.g., age, weight, etc.). For example, for a machine learning model that assesses multiple biometric parameters, a clinician may decide to prioritize hospitalization event recall for one biometric parameter (e.g., heart failure) over another (e.g., weight), and select a lower threshold for heart failure and a higher threshold for weight. As another example, the RMS system may include a plurality of machine learning models, where each machine learning model corresponds to a different biometric parameter or patient characteristic.

Referring to FIG. 12A, based on a determination made by the machine learning model, the system may output, to a user interface device such as user interface device 310 in FIG. 3, a graphical user interface (GUI) 1200 that displays the performance metrics calculated during validation at step 440. The GUI 1200 may display, for example, a curve or graph 1202 depicting a relationship between two metrics, such as alert frequency and hospitalization event recall.

Although FIG. 12A depicts a trade-off between hospitalization event recall and alerting frequency metrics, the GUI 1200 may be configured to allow a user to select any two (or more) metrics that are of interest for them to be displayed in the graph 1202. The GUI 1200 may, for example, receive input from a user via a user input device (e.g., mouse, keyboard, touchscreen, etc.) and/or prompt a user for input (e.g., using a pop-up notification, fillable cells, and/or touch buttons) regarding which metrics to display on the graph 1202. Alternatively or in addition thereto, the GUI 1200 may display a chart similar to the table exemplified in FIG. 11.

The GUI 1200 may enable a user to evaluate trade-offs of two or more metrics (e.g., two or more metrics selected by the user) and to input or select a threshold based on acceptable trade-offs and/or compromises to the user. The GUI 1200 may prompt and/or enable a user to input a desired threshold (or alternatively, a desired alerting frequency, hospitalization recall, or another desired metric) for the machine learning system. The selected threshold may then be displayed on the GUI 1200 (e.g., with an explanation 1204), and the machine learning system may apply the threshold to current and/or incoming data (e.g., current and/or incoming readings data due to monitoring patients) to decide whether to raise an alert.

The explanation 1204 may include a comparison with a default or original alerting system used by the user (e.g., a default or initial alerting system used by a hospital or another institution). For example, the explanation 1204 may indicate that using the selected threshold with the machine learning system will result in a certain percentage higher hospitalization event recall and a certain percentage lower alert frequency. As another example, the explanation 1204 may include a comparison with a default threshold, last threshold selected by the user, and/or a threshold determined by the machine learning system. Based on the provided information, the user may use the current, chosen threshold (e.g., “USE CHOSEN THRESHOLD”), or revert to the default or original threshold or a threshold corresponding to a default or previous metric (e.g., “MATCH ORIGINAL RECALL” and/or “MATCH ORIGINAL ALERTING FREQUENCY”). For example, the GUI 1200 may include selectable icons or buttons (e.g., on a touch screen and/or selectable via a mouse) that allow the user to make these selections. Although recall and alerting frequency are shown as examples, the user might also choose to select a previous or default threshold (e.g., “USE DEFAULT THRESHOLD” or “USE LAST THRESHOLD”), to select user-set presets (e.g., “USE THRESHOLD 1”) or to choose a threshold corresponding to another default, previous, or pre-set metric, such as hospitalization event recall, alert precision, alert recall, and/or an alert F1-score.

As an example, acting on a large number of alerts may not be possible depending on an availability of clinicians and/or other staff. In such a case, the threshold may be adjusted (e.g., by a user and/or by the machine learning system based on received information such as staffing information) to keep a total number of alerts at a level that the clinicians are able to handle at their availability, which may lead to a lower alerting frequency, but could also lead to a lower hospitalization event recall, depending on the learned optimal combinations and/or weightings by the machine learning system. In contrast, if the clinicians' availability is high, then the threshold could be lowered so that more alerts may be raised, which may increase a number of patients receiving attention and may increase the hospitalization event recall. In some examples, the machine learning system may track outcome information (e.g., treatment information for a patient receiving an alert and/or discharge date) and refine the weighting of the plurality of algorithms and/or refine the threshold based on the outcome information (for example, to optimize a quality of care or to reduce a patient's time spent at the hospital, etc.).

FIG. 12B illustrates a method 1250 of training a model to predict hospitalization event recall for a patient based on a current reading. FIG. 12C illustrates a method 1270 used by the machine learning model during training.

Referring to FIG. 12A, the method 1250 may include, at step 1252, receiving a plurality of readings for a plurality of patients. The plurality of readings may include a plurality of biometric readings for a plurality of biometric parameters over a period of time. The method 1250 may include, at step 1254, receiving additional patient information for each of the plurality of patients. The additional patient information may include demographic information (e.g., weight, age, gender, height, body mass index or BMI, etc.), location, comorbidities (e.g., comorbidities at a time of a reading), current or past medications, diagnoses, treatment history or information, care programs a patient is currently enrolled in, physician notes, patient intake data, medical history, recent lab results, previous health events encountered, hospital admissions data, demographic and/or clinical metadata, etc.

To train a model for a specific population, the method 1250 may include, at step 1256, filtering the plurality of readings based on the additional information. For example, to train a model for heart failure, filtering at step 1256 may include selecting the plurality of readings associated with patients identified as being hospitalized for and/or diagnosed with heart failure or heart related diseases. Alternatively or in addition thereto, filtering at step 1256 may include filtering the plurality of readings based on biometric parameters to, for example, train a model specific to a certain type of reading, such as blood sugar reading. Aspects disclosed herein may be used to customize a trained model based on population, demographic information, location, disease, biometric parameters, etc.

The method 1250 may include, at step 1258, identifying a ground truth label for each of the plurality of readings. For example, identifying the ground truth label at step 1258 may include receiving ground truth labels or assignments for each reading, such as true or false, or 1 or 0. As another example, identifying the ground truth label at step 1258 may include determining the ground truth label based on a predetermined policy, such as determining whether the reading occurred in a calculated admission window and/or using the method described with respect to FIG. 7. The ground truth labels may be based on hospitalization events.

The method 1250 may include, at step 1260, applying a plurality of algorithms to each of the plurality of readings to output a plurality of scores for each reading. Each reading may receive a score or indication (e.g., alert or no alert, 1 or 0) from each algorithm. For example, each algorithm may determine, for each reading, a probability that a patient will be hospitalized or need treatment based on the reading.

The method 1250 may include, at step 1262, training a machine learning model using the plurality of output scores and the identified ground truth labels. The machine learning model may be trained to learn a weighting of the plurality of algorithms and/or to predict, based on the output scores, indications that target the ground truth labels. The machine learning model may be trained to take, as input, a plurality of readings and determine or predict, as output, an aggregate score (e.g., aggregate probability) and/or an overall indication of whether an alert should be raised. The method 1250 may include, at step 1264, saving the trained machine learning model to electronic or digital storage.

Referring to FIG. 12C, from the perspective of a machine learning model being trained, a method 1270 of training may include, at step 1272, receiving a plurality of scores for each of a plurality of readings. The plurality of scores for each reading may have been output by a plurality of algorithms, respectively, applied to the plurality of readings. The plurality of scores may represent probabilities of hospitalization determined by the plurality of algorithms. The plurality of scores may be received in a matrix that identifies, for each reading, each algorithm and its output score. The matrix may include additional information, such as population, age, gender, etc.

The method 1270 may include, at step 1274, receiving a ground truth label for each of the plurality of readings. The ground truth label for a reading may correspond to whether a patient associated with the reading was hospitalized within a certain time frame after the reading and/or the reading occurred within a calculated admission window of an admission date (e.g., a predetermined number of n days before an admission date and/or an admission window calculated as described with reference to FIG. 7). The ground truth labels may be received in a matrix identifying, for each reading, the ground truth label or assignment.

The method 1270 may include, at step 1276, using the received plurality of scores and the identified ground truth labels to learn a weighting and/or combination of the plurality of algorithms. The identified ground truth labels may be used as target outputs. The machine learning model may be trained to receive, as input, a plurality of readings and/or scores for each reading and determine, as output, an aggregate score for each reading. The machine learning model may also calculate an aggregate score for each type of reading (e.g., based on biometric parameter) and/or for each patient. The method 1270 may include saving the learned weighting and/or combination to electronic or digital storage.

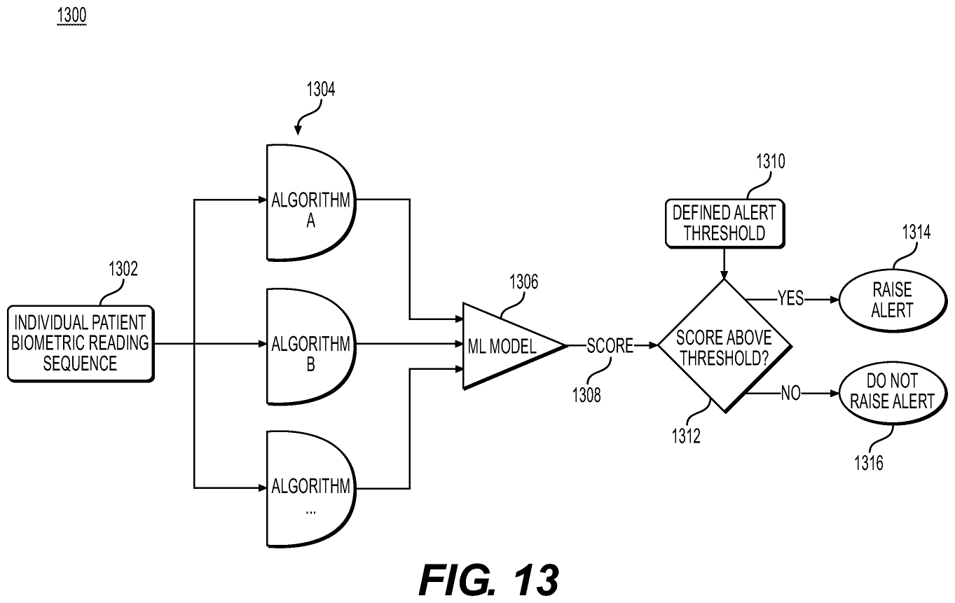

FIG. 13 depicts a method for using a machine learning system to determine whether to raise an alert, according to an exemplary embodiment. Referring to FIGS. 4 and 13, during use, a remote patient monitoring (RMS) system 1300 may, at step 460, receive an individual biometric reading sequence 1302 (e.g., from the individual reading sequence 404). The individual biometric reading sequence 1302 may include one or more readings for one or more biometric parameters. For example, the individual biometric reading sequence 1302 may include historical reading data, in addition to a current reading or a reading of the day. The RMS system 1300 may also receive additional patient data (e.g., additional patient information 906 described with reference to FIGS. 9A-9B).

An alerting algorithms suite 1304 may include a plurality of algorithms (e.g., Algorithm A, Algorithm B, and/or Algorithm C, etc.) each incorporating or using different logic. Each individual algorithm of the alerting algorithms suite 1304 may analyze the one or more readings of the individual biometric reading sequence 1302 (optionally with the additional patient information) and output a determination (e.g., True or False) indicating a prediction of a hospitalization event and/or whether an alert should be raised.

A trained machine learning model 1306 (e.g., classification model) may receive the outputs of the alerting algorithms suite 1304. In some examples, where an individual output is False, the machine learning model 1306 may not receive the output and infer from an absence of the output that the determination is False.

The machine learning model 1306 may use the learned optimized combinations and/or weightings (e.g., as learned at step 430) to determine an aggregate or final score or value 1308. The machine learning model 1306 may determine one aggregate score 1308 for all readings for a patient for each measured biometric parameter (e.g., one aggregate score 1308 for a first biometric parameter, such as a blood sugar reading, one aggregate score 1308 for a second biometric parameter, such as a weight reading, one aggregate score 1308 for a third biometric parameter, such as a temperature reading, etc.), and/or one aggregate score 1308 for the patient for all (e.g., first, second, and third) biometric parameters. The machine learning model 1306 may also consider additional patient information (e.g., additional patient information 906 described with reference to FIGS. 9A-9B) to determine the aggregate score 1308. Alternatively or in addition thereto, the alerting algorithms suite 1304 may consider the additional patient information in the individual determinations and/or outputs.

The RMS system 1300 (or the machine learning model 1306) may receive a defined or predetermined alert threshold 1310, which may have been determined at step 450 of receiving user input. As previously described, the defined alert threshold 1310 may be input by a user (e.g., using GUI 1200 described with reference to FIG. 12) and/or determined by the machine learning model 1306 based on other inputs (e.g., desired alerting frequency and/or staffing availability). For example, the predetermined alert threshold 1310 may be an optimized score threshold based on various inputs received at step 450 and/or available in a patient database or in historical data sequences 402.

Applying the alert score to a threshold at step 470 may include determining whether the aggregate score 1308 is above (or alternatively, at or above, below, or at or below) the defined alert threshold 1310, as indicated by determination 1312. The machine learning model 1306 (or alternatively, another model or processor of the RMS system 1300) may make determination 1312.

If the RMS system 1300 determines that the aggregate score 1308 is above the defined alert threshold 1310 (“Yes”), then the RMS system 1300 may raise or output an alert 1314. The alert 1314 may be a notification on a device (e.g., remote device, computer, phone, pager, tablet, etc.) carried by a clinician, practitioner, or another staff member, a notification on a patient device, a notification on a hospital or institution device (e.g., patient monitor or device that is monitoring biometric parameters of the patient and in communication with RMS 1300, such as a heart rate monitor, a thermometer, pulse monitor, electrocardiogram or EKG monitor, etc.), an alarm system (e.g., a sound alarm and/or a blinking light), etc. Aspects disclosed herein are not limited to an implementation of an alarm or notification. If the RMS system 1300 determines that the aggregate score 1308 is not above the defined alert threshold 1310 (“No”), then the RMS system 1300 may make a determination 1316 to not raise an alert. Alternatively or in addition thereto, the RMS system 1300 may determine to output a notification or store a result indicating that an alert was not raised.

In addition to outputting the alert 1314 and/or determination to not alert 1316, the RMS system 1300 may output the determined aggregate score 1308 at step 480 of providing the aggregate alert score to the user. The user may remember the defined threshold 1310 and, if circumstances have changed, may evaluate the output aggregate score 1308 and, even though an alert was raised, deprioritize an action to be taken regarding the patient. For example, if a hospital has become unusually busy but the defined alert threshold 1310 has not yet been updated or adjusted, the user may make a determination based on the output aggregate score 1308 rather than the fact that the RMS system 1300 output an alert 1314.

The RMS system 1300 may analyze a plurality of biometric readings for a patient, and determine an aggregate score 1308 for each biometric reading. If the RMS system 1300 determines to raise an alert for at least one aggregate score 1308, providing the aggregate alert score at step 480 may include providing and/or outputting a list or ranking of all aggregate scores 1308 and their associated biometric parameters for a patient. The list may rank all aggregate scores 1308 from highest to lowest, and a clinician may assess, from the list or ranking, issues to prioritize for a patient (e.g., blood sugar, temperature, etc.). The RMS system 1300 may also use additional information or policies to determine an order of the ranking (e.g., such as placing certain conditions high when their aggregate scores 1308 were above the threshold, such as heart rate or blood sugar). In some examples, the list may omit biometric parameters where the aggregate score 1308 was not above the predefined threshold.

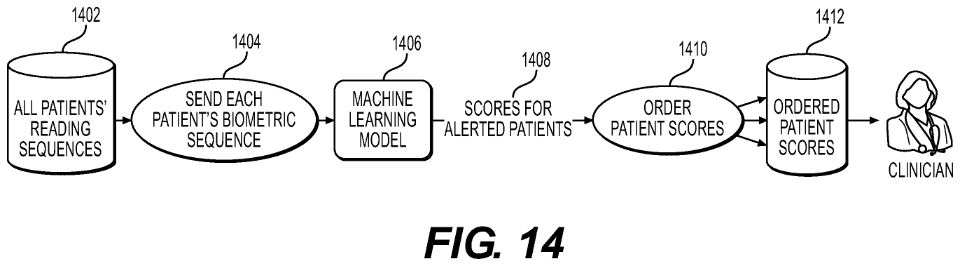

Referring to FIG. 14, determining and providing the aggregate score 1308 at steps 460, 470, and 480 may be performed for a plurality of patients (e.g., at a site such as a hospital). The site may monitor, collect, and/or store all patients' reading sequences 1402 through various measurement devices, databases, or other storage and measurement systems. Within the patient reading sequences 1402, individual reading sequences 1404 may be fed to a machine learning model 1406 (e.g., a trained alert prediction system), which may determine aggregate scores 1408 for all patients. Each aggregate score 1408 may be associated with one patient, and may reflect a probability (e.g., of hospitalization and/or of an alert) based on all of one or more measured biometric parameters. Alternatively, each patient may have a plurality of aggregate scores 1408 associated with each of their measured biometric parameters. Where the machine learning model 1406 evaluates just one biometric parameter or one group of related biometric parameters, the machine learning model 1406 may output one biometric parameter for one patient.

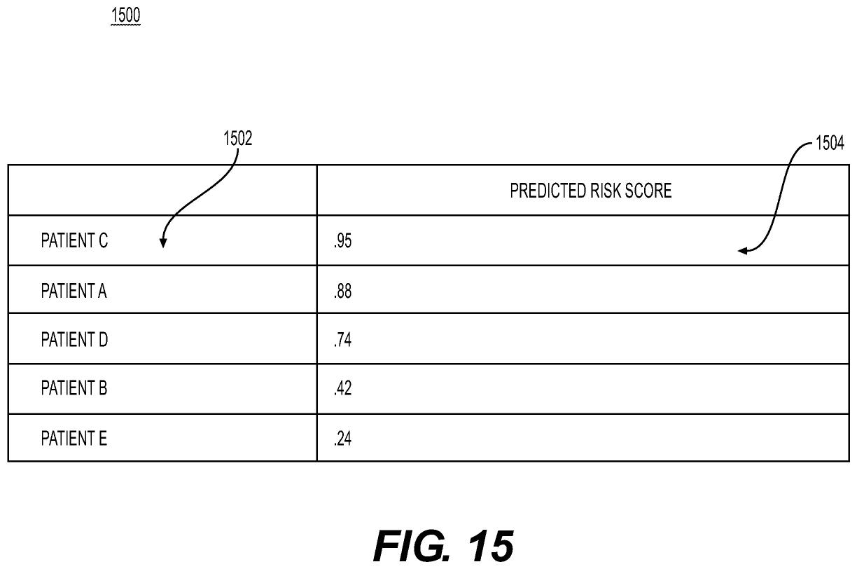

The machine learning model 1406 may determine an order or ranking 1410 of the patients and/or their scores (e.g., in descending order), and provide an output 1412 of the order (e.g., a list on a display) to the clinician. Referring to FIG. 15, a display 1500 of the ordered/ranked patients may be provided via a graphical user interface. The display 1500 may indicate identifying information 1502 of the patient (e.g., name, patient ID, room number, etc.) and the determined aggregate scores 1504 for each patient. The display 1500 may show the identifying information 1502 and aggregate scores 1504 in the determined order (e.g., descending order). The display 1500 may also show other additional patient information, such as demographic information, so that a clinician may interpret how each patient's score compares to other patients in their population. Alternatively or in addition thereto, the machine learning model 1406 may determine a priority value based on the aggregate score 1408 and/or additional information (e.g. inputs by a clinician and/or based on additional patient information), and the display 1500 may order the patient's based on the determined priority. In some examples, where the machine learning system 1406 assesses multiple biometric parameters for each patient, the display 1500 may also indicate a biometric parameter contributing most to the aggregate score 1408 and/or priority level for each patient.

Referring to FIGS. 4, 13-14, and 16A-16B, the system may provide one or more displays 1602, 1604 that include one or more explanations 1606, 1608, respectively, at step 490 of outputting an explanation. The explanations 1606, 1608 may include a list or ranking of the individual algorithms in the suite of algorithms 1304 that were weighted most and/or had a highest contribution to the aggregate score 1408 for an individual patient so that a user (e.g., clinician) may quickly identify concerning patterns in the individual patient's data and intervene to rectify an underlying issue.

Referring to FIG. 17, the RMS system (e.g., via machine learning model 1406) may perform a method 1700 of determining an explanation for an aggregate alert score. The method 1700 may include, at step 1702, determining a weight of each algorithm among a plurality of algorithms and/or determining a weight of each feature among a plurality of features. Alternatively or in addition thereto, step 1702 may include determining a weight of each biometric parameter where readings for multiple biometric parameters are received. Determining a weight of each feature or algorithm at step 1702 may include using global feature importance for an overall model or by using explanation frameworks, such as “Explain Like I'm Five” (ELI5) or local interpretable model agnostic explanations (LIME), to calculate feature importance for a particular prediction.

The method 1700 may include, at step 1704, ranking the algorithms and/or features according to the determined weights. The algorithms may be ranked in descending order. Alternatively, the algorithms may be ranked in ascending order.

The method 1700 may include, at step 1706, filtering certain algorithms and/or features from the ranking. Alternatively, the method 1700 may include filtering algorithms before ranking the algorithms. Filtering at step 1706 may include removing algorithms or features that did not have a score indicating a high probability of hospitalization and/or a high alert (e.g., a score below a predetermined filtering threshold) and/or selecting algorithms or features that had a score indicating a high probability of hospitalization and/or alert (e.g., a score above a predetermined filtering threshold). In addition, if the model was trained to ensure a sparsity of features (e.g., using L2 regularization), algorithms and/or features that had a smaller coefficient (e.g., an absolute value that is less than a predetermined number, which may be close to zero) may be removed from the ranking at step 1706. Algorithms and/or features may also be filtered at step 1706 based on demographic or clinical metadata. For example, predetermined metadata features (e.g., biometric parameters, population type, diagnosis) and/or metadata features with high importance may be selected. These metadata features may be visualized differently than alerting algorithm features.

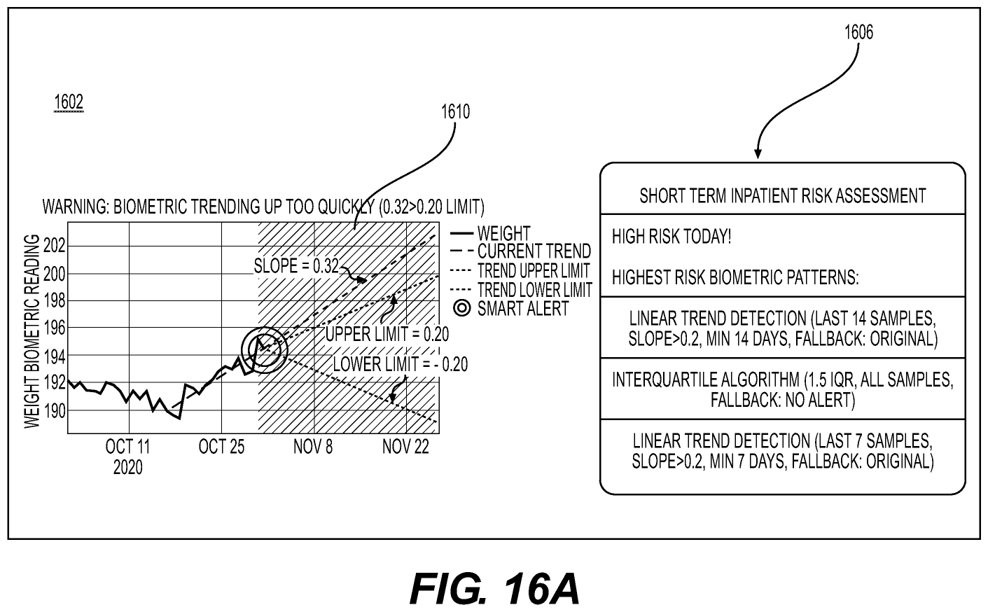

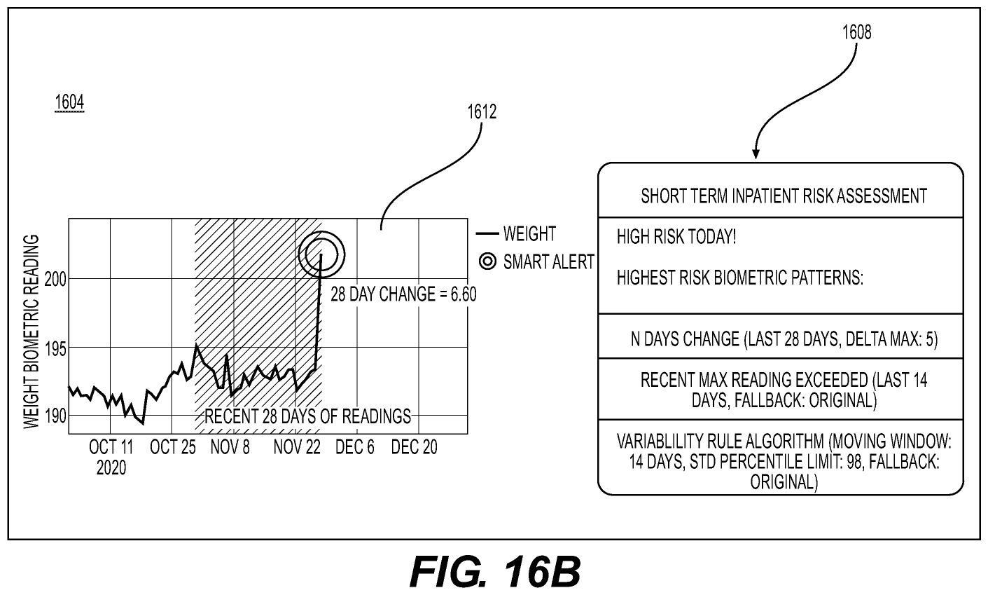

The method 1700 may include, at step 1708, outputting a graphical user interface (GUI) that includes a visual explanation based on the filtered ranking. For example, outputting an explanation at step 1708 may include outputting a list, in order of the filtered ranking, of the algorithms that contributed most (e.g., top one, two, or three algorithms) to a determination (e.g., that an alert should be raised). The GUI may provide an option to select a number of top algorithms to display (e.g., two, three, etc.) and display the algorithms corresponding to the selection and/or on a dashboard. A display of each algorithm may include a graph or chart, such as the graphs or charts exemplified in FIGS. 6A-6C and/or in FIG. 16A or 16B.

For example, referring back to FIGS. 16A and 16B, the display 1602 may indicate readings data (e.g., in a graph or chart 1610) for a patient at a first point in time or reading, and display 1604 may indicate readings data (e.g., in a graph or chart 1612) for the patient at a second or later point in time or reading. The explanation 1606 for display 1602 may indicate different algorithms than the explanation 1608 for display 1604, as different algorithms may have contributed more to the alert based on different biometric patterns.

In addition to charts or graphs 1610, 1612, the displays 1602, 1604 may include visualizations and/or tables that indicate statistics of a comparable population, how a patient's readings and/or demographic or clinical metadata compares with an overall population's statistics or trends, how a patient's readings and/or demographic or clinical metadata compares with a comparable population statistics and/or trends, etc. These charts or graphs 1610, 1612, explanations 1606, 1608, and other data are not limited to the specific displays 1602, 1604 shown. For example, explanations in a form of text may be texted, emailed, and/or printed to a clinician or patient, or displays may be adjusted or adapted for certain devices (e.g., mobile phones, smartwatches, or other mobile devices), etc.

Aspects disclosed herein may be used to determine whether, based on a current reading for a patient, an alert should be raised for a clinician. Referring to FIG. 18, a method 1800 may include, at step 1802, receiving a current reading for one or more biometric parameters. The method 1800 may include, at step 1804, applying a plurality of algorithms to the current reading to output a plurality of scores, respectively, for each reading. Each of the plurality of algorithms may use different logic to evaluate the current reading in view of previous readings.

The method 1800 may include, at step 1806, applying a machine learning model (e.g., alert prediction model) to determine an aggregate score based on a weighting of the plurality of algorithms and the plurality of scores output by the plurality of algorithms. The machine learning model may have been trained based on significant medical events (e.g., hospitalization events or admissions) and/or assigned ground truth labels (e.g., ground truth labels assigned based on hospitalization events).

In some examples, the plurality of algorithms may be filtered before being applied, for example, based on a type of biometric reading and/or based on a predetermined weighting to be applied. In other examples, all algorithms may be applied to the current reading, and later, algorithms that contributed less to the aggregate score may be filtered and/or omitted from the analysis.

The method 1800 may include, at step 1808, comparing the aggregate score to a threshold to determine whether to raise an alert. The threshold may have been predetermined by a user based on various factors presented (e.g., accuracy, alerting frequency, hospitalization event recall, alert precision, alert recall, alert F1-score, etc. during a validation and/or trial period). Alternatively, the threshold may have been determined based on other user input (e.g., desired alert frequency, staffing information, type of institution (e.g., large hospital or small hospital, urgent care clinic, etc.), etc. In some examples, the threshold may have been optimized by a machine learning system. In some examples, comparing the aggregate score to a threshold may include determining that the aggregate score is greater than or equal to the threshold. In other examples, comparing the aggregate score to a threshold may include determining that the aggregate score is greater than the threshold. In some examples, comparing the aggregate score to a threshold may include determining that the aggregate score is less than or equal to the threshold. In other examples, comparing the aggregate score to a threshold may include determining that the aggregate score is less than the threshold. In at least one example, comparing the aggregate score to a threshold may include determining that the aggregate score is equal to the threshold.

The method 1800 may include, at step 1810, outputting an alert and/or the determined aggregate score. For example, if, at step 1808, the aggregate score was determined to be greater than or equal to the threshold, outputting an alert at step 1810 may include providing a notification on a display or other device. The notification may indicate a patient's name, room, etc., the aggregate score, and/or one or more readings or types of biometric parameters contributing to the aggregate score. Alternatively, if, at step 1808, the aggregate score was determined to be less than or equal to the threshold, outputting an alert at step 1810 may include providing a notification on a display or other device. The notification may indicate a patient's name, room, etc., the aggregate score, and/or one or more readings or types of biometric parameters contributing to the aggregate score.

The method 1800 may include, at step 1812, outputting an explanation based on the weighting of the plurality of algorithms based on the weighting of the plurality of algorithms and the aggregate score. Outputting the explanation at step 1812 may include providing a display showing readings data (e.g., trend data) and a list of algorithms (e.g., based on a ranking or weight) that contributed most to the aggregate score and/or indicating determined high risk patterns. In addition, outputting the explanation at step 1812 may include outputting the types of biometric parameters that raised an alert. Where the machine learning model has been trained to consider a plurality of biometric parameters and/or reading types, the aggregate score may be based a composite aggregate score calculated based on each aggregate score for a plurality of biometric parameters. The machine learning model may have been trained to learn an optimal weighting or combination of scores for certain biometric parameters, in addition to an optimal weighting or combination of algorithms. In this case, outputting the explanation at step 1812 may include outputting the biometric parameters (e.g., heart rate) that contributed most to the aggregate score.

Outputting an explanation at step 1812 may include filtering the algorithms and/or selecting a top N algorithms having a highest magnitude or weighting. Filtering may include performing L1 or L2 regularization (e.g., L1 norm regularization). Filtering may include observing feature importance of individual algorithms on a final prediction score, and keeping only the alerting algorithms with a top N highest feature importance values.

The method 1800 may also include receiving outcome information, such as discharge date and/or treatment information, and the machine learning model may be refined based on outcome information. In some examples, the machine learning model may learn patterns for specific (e.g., frequent) patients, and may make refinements based on the specific patient to further individualize a weighting and/or method of raising an alert.

Aspects disclosed herein may be extended to process data from multiple biometric types such as weight, blood sugar, blood pressure, etc. to generate alert predictions. Aspects disclosed herein may be used with different strategies that can be applied to pre-process data from multiple biometrics to prepare the data for input into the machine learning model described herein.

For example, all data samples (e.g., from different biometric types) may be standardized to one sample per day, per biometric, and labels may be generated based on unique days using hospitalization records. Each alerting algorithm may be used on each biometric sequence, and the machine learning algorithm may take, as input, an indication of a biometric and alerting algorithm combination. For example, an input matrix (e.g., model inputs matrices shown in FIGS. 9A and 9B) may include a separate column for each biometric and alerting algorithm combination. The machine learning model may then be trained on this data frame.

As another example, samples from different biometric types may be interleaved in time, and one label may be generated for each sample data point. This example may provide a sparser data but may still capture relations between different biometric types to generate alert predictions.

FIG. 19 illustrates an implementation of a computer system that may execute techniques presented herein. The computer system 1900 can include a set of instructions that can be executed to cause the computer system 1900 to perform any one or more of the methods or computer based functions disclosed herein. The computer system 1900 may operate as a standalone device or may be connected, e.g., using a network, to other computer systems or peripheral devices.

In a networked deployment, the computer system 1900 may operate in the capacity of a server or as a client user computer in a server-client user network environment, or as a peer computer system in a peer-to-peer (or distributed) network environment. The computer system 1900 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 1900 can be implemented using electronic devices that provide voice, video, or data communication. Further, while a single computer system 1900 is illustrated, the term “system” shall also be taken to include any collection of systems or sub-systems that individually or jointly execute a set, or multiple sets, of instructions to perform one or more computer functions.

As illustrated in FIG. 19, the computer system 1900 may include a processor 1902, e.g., a central processing unit (CPU), a graphics processing unit (GPU), or both. The processor 1902 may be a component in a variety of systems. For example, the processor 1902 may be part of a standard personal computer or a workstation. The processor 1902 may be one or more general processors, digital signal processors, application specific integrated circuits, field programmable gate arrays, servers, networks, digital circuits, analog circuits, combinations thereof, or other now known or later developed devices for analyzing and processing data. The processor 1902 may implement a software program, such as code generated manually (i.e., programmed).

The computer system 1900 may include a memory 1904 that can communicate via a bus 1908. The memory 1904 may be a main memory, a static memory, or a dynamic memory. The memory 1904 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 1904 includes a cache or random-access memory for the processor 1902. In alternative implementations, the memory 1904 is separate from the processor 1902, such as a cache memory of a processor, the system memory, or other memory. The memory 1904 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 1904 is operable to store instructions executable by the processor 1902. The functions, acts or tasks illustrated in the figures or described herein may be performed by the programmed processor 1902 executing the instructions stored in the memory 1904. The functions, acts or tasks are independent of the particular type of instructions set, storage media, processor or processing strategy and may be performed by software, hardware, integrated circuits, firm-ware, micro-code and the like, operating alone or in combination. Likewise, processing strategies may include multiprocessing, multitasking, parallel processing and the like.

As shown, the computer system 1900 may further include a display unit 1910, such as a liquid crystal display (LCD), an organic light emitting diode (OLED), a flat panel display, a solid-state display, a cathode ray tube (CRT), a projector, a printer or other now known or later developed display device for outputting determined information. The display 1910 may act as an interface for the user to see the functioning of the processor 1902, or specifically as an interface with the software stored in the memory 1904 or in the drive unit 1906.

Additionally or alternatively, the computer system 1900 may include an input device 1912 configured to allow a user to interact with any of the components of system 1900. The input device 1912 may be a number pad, a keyboard, or a cursor control device, such as a mouse, or a joystick, touch screen display, remote control, or any other device operative to interact with the computer system 1900.

The computer system 1900 may also or alternatively include a disk or optical drive unit 1906. The disk drive unit 1906 may include a computer-readable medium 1922 in which one or more sets of instructions 1924, e.g. software, can be embedded. Further, the instructions 1924 may embody one or more of the methods or logic as described herein. The instructions 1924 may reside completely or partially within the memory 1904 and/or within the processor 1902 during execution by the computer system 1900. The memory 1904 and the processor 1902 also may include computer-readable media as discussed above.

In some systems, a computer-readable medium 1922 includes instructions 1924 or receives and executes instructions 1924 responsive to a propagated signal so that a device connected to a network 1950 can communicate voice, video, audio, images, or any other data over the network 1950. Further, the instructions 1924 may be transmitted or received over the network 1950 via a communication port or interface 1920, and/or using a bus 1908. The communication port or interface 1920 may be a part of the processor 1902 or may be a separate component. The communication port 1920 may be created in software or may be a physical connection in hardware. The communication port 1920 may be configured to connect with a network 1950, external media, the display 1910, or any other components in system 1900, or combinations thereof. The connection with the network 1950 may be a physical connection, such as a wired Ethernet connection or may be established wirelessly as discussed below. Likewise, the additional connections with other components of the system 1900 may be physical connections or may be established wirelessly. The network 1950 may alternatively be directly connected to the bus 1908.

While the computer-readable medium 1922 is shown to be a single medium, the term “computer-readable medium” may include a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that cause a computer system to perform any one or more of the methods or operations disclosed herein. The computer-readable medium 1922 may be non-transitory, and may be tangible.

The computer-readable medium 1922 can include a solid-state memory such as a memory card or other package that houses one or more non-volatile read-only memories. The computer-readable medium 1922 can be a random-access memory or other volatile re-writable memory. Additionally or alternatively, the computer-readable medium 1922 can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, can be constructed to implement one or more of the methods described herein. Applications that may include the apparatus and systems of various implementations can broadly include a variety of electronic and computer systems. One or more implementations described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The computer system 1900 may be connected to one or more networks 1950. The network 1950 may define one or more networks including wired or wireless networks. The wireless network may be a cellular telephone network, an 802.11, 802.119, 802.20, or WiMax network. Further, such networks may include a public network, such as the Internet, a private network, such as an intranet, or combinations thereof, and may utilize a variety of networking protocols now available or later developed including, but not limited to TCP/IP based networking protocols. The network 1950 may include wide area networks (WAN), such as the Internet, local area networks (LAN), campus area networks, metropolitan area networks, a direct connection such as through a Universal Serial Bus (USB) port, or any other networks that may allow for data communication. The network 1950 may be configured to couple one computing device to another computing device to enable communication of data between the devices. The network 1950 may generally be enabled to employ any form of machine-readable media for communicating information from one device to another. The network 1950 may include communication methods by which information may travel between computing devices. The network 1950 may be divided into sub-networks. The sub-networks may allow access to all of the other components connected thereto or the sub-networks may restrict access between the components. The network 1950 may be regarded as a public or private network connection and may include, for example, a virtual private network or an encryption or other security mechanism employed over the public Internet, or the like.

In accordance with various implementations of the present disclosure, the methods described herein may be implemented by software programs executable by a computer system. Further, in an exemplary, non-limited implementation, implementations can include distributed processing, component/object distributed processing, and parallel processing. Alternatively, virtual computer system processing can be constructed to implement one or more of the methods or functionality as described herein.

Aspects disclosed herein may build and/or provide an explainable alerting solution that uses different relatively simple alerting algorithms, readings data, and/or other patient-related data, to decide whether an alert should be produced for a patient considering their recent biometric readings and medical details. Aspects disclosed herein may adjust a prediction process based on a user's (e.g., system owner's or clinician's) feedback and provide transparency by explaining a reasoning or calculation process of a machine learning model so that the user may make more informed decisions. Aspects disclosed herein may provide a highly personalized, automated, and explainable alerting system that aims to reduce a user's (e.g., a clinician's) alert fatigue by only alerting if a patient is at risk of a relevant adverse health event.

Aspects disclosed herein may provide an alert prediction model that uses the capabilities of individual alerting algorithms and is more advanced than the current simple threshold-based methods. Aspects disclosed herein may provide explanations using the contributing alerting algorithms' insights for a generated prediction, which may be more intuitive than the black-box AI/ML models. Aspects disclosed herein may use an estimated risk for hospitalization events as criteria for raising biometric alerts. This is a key factor in reducing alert fatigue by generating more relevant and/or meaningful alerts and pointing clinicians towards patients that are most at risk of an adverse health event. Finally, aspects disclosed herein may enable users (e.g., system-owners) to control a trade-off between different objectives they want to optimize, which may facilitate an engagement of users with the underlying system.

Aspects disclosed herein may provide a machine learning system that learns an optimal combination of different alerting algorithms so as to achieve better performance than any of the single alerting algorithms on their own. Aspects disclosed herein may be adjusted (e.g., threshold, weighting, parameters, etc.) based on a user response or feedback and allow a user to trade-off and/or choose a balance between different objectives to be optimized.

Aspects disclosed herein may provide transparency through explanations that are generated by contributing alerting algorithms to a prediction score, which may be more intuitive than using black-box artificial intelligence or machine learning models.

Aspects disclosed herein may use hospitalization events as a criteria for raising biometric alerts to reduce alert fatigue and point clinicians toward and/or indicate only the most relevant alerts.

In comparison with existing threshold based technologies which generally use a single biometric reading type to predict alerts, aspects disclosed herein may use multiple reading types together in order to generate more accurate alerts. Aspects disclosed herein may use patterns that may depend on multiple biometrics. Aspects disclosed herein may condition a response of an alert prediction model on clinical metadata and patient demographics.

Aspects disclosed herein may use techniques that are different than typical stacking-based ensembling techniques in machine learning, as stacking ensembles may require individual models or algorithms to be trained against a desirable target (i.e., to be supervised). Aspects disclosed herein may use a classifier to balance some heuristic-based (non-supervised) algorithms in predicting a desirable target.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosed embodiments are not limited to any particular implementation or programming technique and that the disclosed embodiments may be implemented using any appropriate techniques for implementing the functionality described herein. The disclosed embodiments are not limited to any particular programming language or operating system.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

The present disclosure furthermore relates to the following aspects.

Example 1. A computer-implemented method for improved provision of health alerts associated with patients, comprising: receiving, by one or more processors, a first reading for a first biometric parameter for a first patient; applying, by the one or more processors, a plurality of algorithms that determine a plurality of first scores, respectively, for the first reading, wherein each of the plurality of algorithms uses different logic; determining, by the one or more processors and using a machine learning model, an aggregate score based on the determined plurality of first scores and on a learned weighting of the plurality of algorithms; comparing, by the one or more processors, the aggregate score to a threshold; and providing, by the one or more processors, an alert to a user based on the comparing.

Example 2. The computer-implemented method of example 1, wherein the machine learning model was trained based at least in part on hospitalization events.

Example 3. The computer-implemented method of any of the preceding examples, wherein each first score indicates a probability of hospitalization based on the first reading.

Example 4. The computer-implemented method of any of the preceding examples, wherein the machine learning model was trained based at least in part on medical events.

Example 5. The computer-implemented method of any of the preceding examples, wherein the machine learning model was trained using a plurality of training readings, wherein each training reading was assigned a ground truth label based on whether the training reading occurred during a predetermined period of time before a medical event.

Example 6. The computer-implemented method of example 5, wherein the predetermined period of time is a calculated admission window, and the medical event is an admission date to a hospital.

Example 7. The computer-implemented method of any of the preceding examples, wherein the user is the first patient or a health care provider.

Example 8. The computer-implemented method of any of the preceding examples, further comprising providing, by the one or more processors, an explanation for the alert based on the learned weighting of the plurality of algorithms and the aggregate score.

Example 9. The computer-implemented method of any of the preceding examples, further comprising: ranking, by the one or more processors, the plurality of algorithms based on a contribution of each algorithm to the aggregate score; and providing a list of algorithms based on the ranking.

Example 10. The computer-implemented method of any of the preceding examples, further comprising: receiving, by the one or more processors, a second reading for a second biometric parameter for the first patient; and applying, by the one or more processors, the plurality of algorithms to determine a plurality of second scores, respectively, for the second reading, wherein the determined aggregate score is further based on the plurality of second scores.

Example 11. The computer-implemented method of any of the preceding examples, further comprising receiving, by the one or more processors, additional information for the first patient, wherein the aggregate score is based on the received additional information.

Example 12. The computer-implemented method of any of the preceding examples, further comprising: receiving, by the one or more processors, a second reading for a second patient; applying, by the one or more processors, the plurality of algorithms that determine a plurality of second scores, respectively, to the second reading; determining, by the one or more processors and using the machine learning model, a secondary aggregate score for the second patient based on the determined plurality of second scores; ranking, by the one or more processors, the aggregate score and the secondary aggregate score; and providing, by the one or more processors, the aggregate score and the secondary aggregate score based on the ranking.

Example 13. The computer-implemented method of any of the preceding examples, wherein the threshold is based on a user input and/or a predetermined alert frequency.

Example 14. A system for improved provision of health alerts associated with patients, the system comprising: a memory having processor-readable instructions stored therein; and a processor configured to access the memory and execute the processor-readable instructions to perform operations comprising: receiving a first reading for a first biometric parameter for a first patient; applying a plurality of algorithms that determine a plurality of first scores, respectively, for the first reading, wherein each of the plurality of algorithms uses different logic; determining, using a machine learning model, an aggregate score based on the determined plurality of first scores and on a learned weighting of the plurality of algorithms; comparing the aggregate score to a threshold; and providing an alert to a user based on the comparing.

Example 15. The system of example 14, wherein the machine learning model was trained based at least in part on medical events.

Example 16. The system of example 14 or 15, wherein each first score indicates a probability of hospitalization based on the first reading.

Example 17. A non-transitory computer-readable medium storing a set of instructions that, when executed by a processor, perform operations for improved provision of health alerts associated with patients, the operations comprising: receiving a first reading for a first biometric parameter for a first patient; applying a plurality of algorithms that determine a plurality of first scores, respectively, for the first reading, wherein each of the plurality of algorithms uses different logic; determining, using a machine learning model, an aggregate score based on the determined plurality of first scores and on a learned weighting of the plurality of algorithms; comparing the aggregate score to a threshold; and providing an alert to a user based on the comparing.

Example 18. The non-transitory computer-readable medium of example 17, wherein the machine learning model was trained based at least in part on medical events.

Example 19. The non-transitory computer-readable medium of example 17 or 18, wherein each first score indicates a probability of hospitalization based on the first reading.

Example 20. The non-transitory computer-readable medium of example 17, 18, or 19, wherein the machine learning model was trained using a plurality of training readings, wherein each training reading was assigned a ground truth label based on whether the training reading occurred during a predetermined period of time before a medical event.

We claim:

1. A computer-implemented method for improved provision of health alerts associated with patients, the method comprising:
receiving, by one or more processors, a first reading for a first biometric parameter for a first patient;
applying, by the one or more processors, a plurality of algorithms that determine a plurality of first scores, respectively, for the first reading, wherein each of the plurality of algorithms uses different logic;
determining, by the one or more processors and using a machine learning model, an aggregate score based on the determined plurality of first scores and on a learned weighting of the plurality of algorithms;
comparing, by the one or more processors, the aggregate score to a threshold;
and
providing, by the one or more processors, an alert to a user based on the comparing.

2. The method of claim 1, wherein the machine learning model was trained based at least in part on hospitalization events.

3. The method of claim 1, wherein each first score indicates a probability of hospitalization based on the first reading.

4. The method of claim 1, wherein the machine learning model was trained based at least in part on medical events.

5. The method of claim 1, wherein the machine learning model was trained using a plurality of training readings, wherein each training reading was assigned a ground truth label based on whether the training reading occurred during a predetermined period of time before a medical event.

6. The method of claim 5, wherein the predetermined period of time is a calculated admission window, and the medical event is an admission date to a hospital.

7. The method of claim 1, wherein the user is the first patient or a health care provider.

8. The method of claim 1, further comprising providing, by the one or more processors, an explanation for the alert based on the learned weighting of the plurality of algorithms and the aggregate score.

9. The method of claim 1, further comprising:
ranking, by the one or more processors, the plurality of algorithms based on a contribution of each algorithm to the aggregate score; and
providing a list of algorithms based on the ranking.

10. The method of claim 1, further comprising:
receiving, by the one or more processors, a second reading for a second biometric parameter for the first patient; and
applying, by the one or more processors, the plurality of algorithms to determine a plurality of second scores, respectively, for the second reading, wherein the determined aggregate score is further based on the plurality of second scores.

11. The method of claim 1, further comprising receiving, by the one or more processors, additional information for the first patient, wherein the aggregate score is based on the received additional information.

12. The method of claim 1, further comprising:
receiving, by the one or more processors, a second reading for a second patient;
applying, by the one or more processors, the plurality of algorithms that determine a plurality of second scores, respectively, to the second reading;
determining, by the one or more processors and using the machine learning model, a secondary aggregate score for the second patient based on the determined plurality of second scores;
ranking, by the one or more processors, the aggregate score and the secondary aggregate score; and
providing, by the one or more processors, the aggregate score and the secondary aggregate score based on the ranking.

13. The method of claim 1, wherein the threshold is based on a user input and/or a predetermined alert frequency.

14. A system for improved provision of health alerts associated with patients, the system comprising:
a memory having processor-readable instructions stored therein; and
a processor configured to access the memory and execute the processor-readable instructions to perform operations comprising:
receiving a first reading for a first biometric parameter for a first patient;
applying a plurality of algorithms that determine a plurality of first scores, respectively, for the first reading, wherein each of the plurality of algorithms uses different logic;
determining, using a machine learning model, an aggregate score based on the determined plurality of first scores and on a learned weighting of the plurality of algorithms;
comparing the aggregate score to a threshold;
and
providing an alert to a user based on the comparing.

15. The system of claim 14, wherein the machine learning model was trained based at least in part on medical events.

16. The system of claim 14, wherein each first score indicates a probability of hospitalization based on the first reading.

17. A non-transitory computer-readable medium storing a set of instructions that, when executed by a processor, perform operations for improved provision of health alerts associated with patients, the operations comprising:
receiving a first reading for a first biometric parameter for a first patient;
applying a plurality of algorithms that determine a plurality of first scores, respectively, for the first reading, wherein each of the plurality of algorithms uses different logic;
determining, using a machine learning model, an aggregate score based on the determined plurality of first scores and on a learned weighting of the plurality of algorithms;
comparing the aggregate score to a threshold;
and
providing an alert to a user based on the comparing.

18. The computer-readable medium of claim 17, wherein the machine learning model was trained based at least in part on medical events.

19. The computer-readable medium of claim 17, wherein each first score indicates a probability of hospitalization based on the first reading.

20. The computer-readable medium of claim 17, wherein the machine learning model was trained using a plurality of training readings, wherein each training reading was assigned a ground truth label based on whether the training reading occurred during a predetermined period of time before a medical event.
